SEQUENCE LISTING (18 sequences)

SEQ ID NO: 1 - protein, 8 aa, Artificial Sequence
  Synthetic polypeptide
GGPLGLAL

SEQ ID NO: 2 - protein, 8 aa, Artificial Sequence
  Synthetic polypeptide
GPLGLAGK

SEQ ID NO: 3 - protein, 4 aa, Artificial Sequence
  Synthetic polypeptide
GPLG

SEQ ID NO: 4 - protein, 19 aa, Artificial Sequence
  Synthetic polypeptide
GPLGLAGKWAAAAKAAAAK

SEQ ID NO: 5 - protein, 19 aa, Artificial Sequence
  Synthetic polypeptide
GPLGLAGKWAAAAKAAAAK

SEQ ID NO: 6 - protein, 9 aa, Artificial Sequence
  Synthetic polypeptide
EGPLGLAGK

SEQ ID NO: 7 - protein, 5 aa, Artificial Sequence
  Synthetic polypeptide
EGPLG

SEQ ID NO: 8 - protein, 7 aa, Artificial Sequence
  Synthetic polypeptide
GALGLPG

SEQ ID NO: 9 - DNA, 19 bp, Artificial Sequence
  Synthetic polynucleotide
tcgcacccagtctctctcc

SEQ ID NO: 10 - DNA, 22 bp, Artificial Sequence
  Synthetic polynucleotide
atcagaaagctaggtgtgtgtt

SEQ ID NO: 11 - DNA, 9 bp, Artificial Sequence
  Synthetic polynucleotide
tcgcaccca

SEQ ID NO: 12 - DNA, 20 bp, Artificial Sequence
  Synthetic polynucleotide
agagtcatgtccaggcagtg

SEQ ID NO: 13 - DNA, 20 bp, Artificial Sequence
  Synthetic polynucleotide
tctcagtacaggtccgtcac

SEQ ID NO: 14 - DNA, 15 bp, Artificial Sequence
  Synthetic polynucleotide
tccgagccggtcgaa

SEQ ID NO: 15 - DNA, 32 bp, Artificial Sequence
  Synthetic polynucleotide
ggagagagatccgagccggtcgaagggtgcga

SEQ ID NO: 16 - DNA, 32 bp, Artificial Sequence
  Synthetic polynucleotide
agcgacagatccgagccggtcgaagtcgtgag

SEQ ID NO: 17 - DNA, 22 bp, Artificial Sequence
  Synthetic polynucleotide
cctctcacgacagtctgtcgtc

SEQ ID NO: 18 - DNA, 22 bp, Artificial Sequence
  Synthetic polynucleotide
ccttcgcacccagtctctctcc

(19) United States
(12) Patent Application Publication
(10) Pub. No.: US 2017/0000909 A1
(43) Pub. Date: Jan. 5, 2017

(54) ENZYME DIRECTED ASSEMBLY OF PARTICLE THERANOSTICS

(71) Applicant: The Regents of the University of California, Oakland, CA (US)

(72) Inventors: Nathan C. Gianneschi, San Diego, CA (US); Michael Hahn, San Diego, CA (US); Robert Mattrey, San Diego, CA (US)

(21) Appl. No.: 15/211,995

(22) Filed: Jul. 15, 2016

Related U.S. Application Data
(62) Division of application No. 14/125,273, filed on Feb. 28, 2014.
(60) Provisional application No. 61/495,841, filed on Jun. 10, 2011.

(51) Int. Cl. A61K 49/12; A61K 49/18; A61K 47/48; A61K 31/704; A61K 31/7048; A61K 49/14; A61K 49/00

(52) U.S. Cl. CPC A61K 49/128; A61K 49/14; A61K 49/1809; A61K 49/0054; A61K 49/0056; A61K 49/0082; A61K 49/0043; A61K 49/0041; A61K 31/704; A61K 31/7048; A61K 47/48338; A61K 47/4823

(57) ABSTRACT

Provided herein, inter alia, are compositions, methods of detecting a diseased tissue in a subject, and methods of treating a subject having cancer.

### CROSS-REFERENCES TO RELATED APPLICATIONS

This application is a divisional of U.S. application Ser. No. 14/125,273, filed Feb. 28, 2014, which is the U.S. National Stage of PCT/US2012/041939, filed Jun. 11, 2012, which claims priority to U.S. Provisional Application No. 61/495,851, filed Jun. 10, 2011, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support from Grant No. FA9550-11-1-0105, awarded by the U.S. Air Force Office of Scientific Research; Grant No. W911NF-11-0264, awarded by the Army Research Office; and Grant Nos. 1R01EB011633 and 1DP20D008724, awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FILE

The Sequence Listing written in file 48537-565D01US_ST25.TXT, created on Jul. 14, 2016, 4,928 bytes, machine format IBM-PC, MS Windows operating system, is hereby incorporated by reference.

### BACKGROUND OF THE INVENTION

There is an ever-increasing knowledge base concerning the molecular signatures of specific diseases and their potential in personalized medicine; however, the translation of this information into clinical practice lags significantly behind. “Theranostic” agents are of particular interest since they combine in vivo imaging for diagnostics and therapeutics within a single system.

There is a tremendous need for novel and effective approaches to molecular imaging in vivo, since current structural imaging techniques do not capitalize on the molecular basis of disease to add specificity. While structure imaging is oftentimes sufficient to answer general clinical questions, it has been inadequate in assessing molecular characteristics of diseased tissues (i.e., tumors). At times, structural imaging techniques are unable to discern benign from malignant tissue, such as lymph nodes or lung nodules. The methods and compositions described herein can fill the void and thus expand the reach of therapy by allowing the visualization, characterization, and measurement of biological processes at the molecular and cellular levels.

### BRIEF SUMMARY OF THE INVENTION

Described herein are programmable stimuli responsive nanomaterials for detecting and treating disease. Further provided is an in vivo assembly of nanoscale objects of specific size, shape, photophysical, magnetic, and pharmacokinetic properties in response to disease-associated enzymatic signals. This approach has been termed Enzyme-directed Assembly of Particle Theranostics (EDAPT). Described herein are methods of making and using DNA-programmable and peptide-programmable materials capable of accumulating and subsequently activating in diseased tissue while evading non-specific accumulation. The present disclosure will alleviate problems limiting the efficacy of current delivery strategies for both diagnostics and therapeutics. Provided herein is a novel diagnostic and therapeutic system directed at diseased tissues.

Aberrantly high activity of pro-oncogenic enzymes is one significant hallmark among the multitude of changes present in tumors. Many such overactive enzymatic biomarkers have been identified but using their presence in diagnostic or therapeutic strategies has lagged behind their discovery. One particularly promising enzyme family, the matrix metalloproteinases (MMPs), has been extensively studied in all phases of cancer progression. These enzymes contribute to oncogenesis and metastasis through several mechanisms and their activity has been observed to increase dramatically as the tumor becomes more aggressive. Increased MMP expression has also been shown to correlate significantly with prognosis in a wide range of malignancies. Described herein is use of molecular imaging of MMP activity as a potential diagnostic, prognostic, and treatment stratification tool. Molecular imaging of MMP activity can be utilized in a similar fashion as FDG-PET, currently used for non-invasive monitoring of tumor molecular activity in patients. The presently disclosed methods and compositions provide clinicians a new means to determine if a given treatment regimen is active at early time points in each specific patient, thus facilitating personalized medicine. Provided herein is a novel strategy for the molecular imaging of MMP activity in vivo that can enable specific and sensitive MMP detection in various cancers.

Provided herein is a method of detecting a diseased tissue in a subject comprising:(a) obtaining a hydrophilic polymer probe comprising: a hydrophilic polymer containing an enzyme cleavable moiety, and a visualizable label; (b) administering a hydrophilic polymer probe to the tissue in an amount sufficient to provide a detectable image; (c) allowing cleavage of a hydrophilic polymer probe by an enzyme, wherein the cleaved hydrophilic polymer probe self-assembles into an amphiphilic polymer aggregate, comprising of amphiphilic polymer and a visualizable label, at the cellular location to be imaged; and (d) imaging the tissue, wherein a detectable image of the self-assembled particle aggregate in the tissue indicates the presence of a diseased tissue. The enzyme cleavable moiety is a peptide-based structure or a DNA-based structure. The visualizable label is a fluorophore, quencher, Gd³⁺ reporter or combinations thereof.

Provided herein is a method of detecting a cancerous tissue in a subject comprising:(a) obtaining a hydrophilic polymer probe comprising: a hydrophilic polymer containing MMP-cleavable polypeptide, and a visualizable label; (b) administering a hydrophilic polymer probe to the tissue in an amount sufficient to provide a detectable image; (c) allowing cleavage of a hydrophilic polymer probe by a cancer-associated enzyme, wherein the cleaved hydrophilic polymer probe self-assembles into an amphiphilic polymer aggregate, comprising of amphiphilic polymer and a visualizable label, at the cellular location to be imaged; and (d) imaging the tissue, wherein a detectable image of the self-assembled particle aggregate in the tissue indicates the presence of a cancerous tissue. The cancer-associated enzyme is a nuclease (endonuclease, exonuclease), a protease, or a matrix metalloprotease (MMP-2, MMP-9).

Provided herein is a method of treating a subject having cancer, the method comprising: (a) obtaining a hydrophilic polymer probe comprising: a hydrophilic polymer containing an enzyme cleavable moiety, targeting structure, and a therapeutic agent; (b) administering a hydrophilic polymer probe to the tissue in an amount sufficient to provide a therapeutic dosage to the subject; (c) allowing the localization of a hydrophilic polymer probe to the targeted tissue in the subject as directed by the targeting polypeptide; and (d) allowing cleavage of a hydrophilic polymer probe by an enzyme, wherein the cleaved hydrophilic polymer probe self-assembles into an amphiphilic polymer aggregate, comprising of amphiphilic polymer and a therapeutic agent, at the cellular location to be treated, thereby treating a subject with cancer. The enzyme cleavable moiety is a peptide-based structure or a DNA-based structure.

Provided herein is a method of treating a subject having cancer, the method comprising: (a) obtaining a hydrophilic polymer probe comprising: a hydrophilic polymer containing an MMP-cleavable polypeptide, targeting structure, and a therapeutic agent; (b) administering a hydrophilic polymer probe to the tissue in an amount sufficient to provide a therapeutic dosage to the subject; (c) allowing the localization of a hydrophilic polymer probe to the targeted tissue in the subject as directed by the targeting polypeptide; and (d) allowing cleavage of a hydrophilic polymer probe by an enzyme, wherein the cleaved hydrophilic polymer probe self-assembles into an amphiphilic polymer aggregate, comprising of amphiphilic polymer and a therapeutic agent, at the cellular location to be treated, thereby treating a subject with cancer. The targeting structure is a peptide-based structure or a nucleic acid-based structure.

Provided herein is an enzymatically-directed self-assembled amphiphilic polymer aggregate comprising: an amphiphilic polymer, which comprises of a hydrophilic cryptic-amphiphile with a cleaved MMP-specific polypeptide, and a labeling group.

Provided herein is an enzymatically-directed self-assembled amphiphilic polymer aggregate comprising: an amphiphilic polymer, which comprises of a hydrophilic cryptic-amphiphile with a cleaved nucleic acid structure, and a labeling group.

Provided herein is a hydrophilic polymer probe, which is prepared by a preparation method comprising synthesizing a polymer using the method of ring-opening metathesis polymerization, and incorporating labeling groups, therapeutic agents and/or targeting groups into said synthesized polymer.

Provided herein is a method of treating a diseased tissue of a subject's body, comprising providing a therapeutic agent conjugated to hydrophophilic polymer probe in a manner to direct self-assembly of the hydrophilic polymer into amphiphilic polymer aggregates and delivery of the drug to the diseased tissue.

Provided herein is a method of treating a MMP-mediated cancerous tissue of a subject's body, comprising providing a therapeutic agent conjugated to hydrophophilic polymer probe in a manner to direct self-assembly of the hydrophilic polymer into amphiphilic polymer aggregates and delivery of the drug to the MMP-mediated cancerous tissue. The MMP-mediated cancerous tissue is a MMP-mediated malignancy, sarcoma, or metastasis.

Presented herein is a method of imaging a diseased tissue of a subject's body, comprising providing a labeling agent conjugated to hydrophophilic polymer probe in a manner to direct self-assembly of the hydrophilic polymer into amphiphilic polymer aggregates and delivery of the labeling agent to the diseased tissue.

Presented herein is a method of imaging a MMP-mediated cancerous tissue of a subject's body, comprising providing a labeling agent conjugated to hydrophophilic polymer probe in a manner to direct self-assembly of the hydrophilic polymer into amphiphilic polymer aggregates and delivery of the labeling agent to the MMP-mediated cancerous tissue.

### BRIEF DESCRIPTION OF THE DRAWINGS

FIGS. 1A-1B show enzyme-directed assembly of particle theranostics. FIG. 1A shows the formation of self-assembled nanoparticles from peptide-polymer amphiphile following cleavage by the enzyme MMP. FIG. 1B shows the use of self-assembled nanoparticles to target tumor tissue in the body.

FIG. 2 illustrates the pharmocokinetic profile and detection properties of an enzyme-responsive micellar nanomaterial.

FIG. 3 illustrates cryptic-amphiphile structures (SEQ ID NOS:3, 6, 7 and 8).

FIGS. 4A-4C show formation of a Type-2 cryptic-amphiphile. FIG. 4A: FIG. 4A shows a pure peptide, directly labeled fluorogenic substrate used for conjugation to a NHS-norborene polymer. FIG. 4B: ESI-MS of peak. FIG. 4C: Fold fluorescence increase as a function of time.

FIGS. 5A-5B illustrate nanoparticle self-aggregation in response to enzyme cleavage of peptide substrates (SEQ ID NOS:3, 8). FIG. 5A: SEQ ID NO:3. FIG. 5B: SEQ ID NO:8.

FIG. 6 shows a schematic diagram of dye labeled self-assembled aggregates. Nanoparticles were designed to generate a unique FRET signal with fluorescein-polymers and rhodamine-polymers.

FIG. 7 shows the FRET signal at an appropriate concentration for the synthesis reaction.

FIG. 8 shows a study to determine the optimal micelle concentration for a FRET based nanoparticle. A concentration of the amphiphile is needed to create particle to unimer equilibrium.

FIG. 9 shows that MRI-agent chelates for direct incorporation into polymer backbone.

FIG. 10 shows multienzymatic responsive micellular nanoparticles.

FIGS. 11A-11L show phosphorylation and dephosphorylation in reversible cycling of particle morphology. FIGS. 11A-11D show that the phosphorylated substrate adopts an aggregate morphology (FIGS. 11A, 11C) and the dephosphorylated substrate does not (FIGS. 11B, 11D). Phosphorylation of the substrate is induced by PKA. FIGS. 11E-11L show proteolysis at the particle shell. Proteolysis of the MMP substrate is controlled by MMP2. M2 micelles form aggregates in the presence of MMP2 while M1 micelles do not.

FIG. 12 illustrates an assembly of peptide-polymer amphiphiles (PPAs) to generate fluorogenic micellar nanoparticles. Polymers are labeled with peptides (SEQ ID NO:4) and dyes, post-polymerization with block sizes determined by SEC-MALS analysis and spectroscopy. Degree of dye incorporation (m), was between 1 and 2 for both PPA-1 and PPA-2.

FIGS. 13A-13D illustrate TEM, DLS and fluorescence spectroscopy of fluorogenic micelles. FIG. 13A shows TEM of 30 nm M3. FIG. 13B shows DLS of M1, M2 and M3 showing hydrodynamic diameters (Dh) in the range of 30-40 nm. FIG. 13C shows fluorescence emission spectra of M1, M2 and FRET-micelle, M3 upon excitation at 470 nm. FIG. 13D shows the ratio of normalized emission intensity for maxima at 563 nm (rhodamine) and 512 nm (fluorescein) over a range of concentrations of PPA-1 and PPA-2 upon excitation at 470 nm. Arrow indicates onset of detectable FRET.

FIGS. 14A-14B illustrate the time-domain fluorescence intensity decay analysis of M1 and M3 for determination of structural parameters. FIG. 14A shows the fluorescence lifetime of M3 and M1 fit to a distance distribution function, and single exponential respectively giving tD=3.98+/−0.01 ns (from M1 data) for unquenched fluorescein. FIG. 14B shows for M3, a range of distances between donor and acceptor (D and A) is considered, expressed as a probability function P(r). The mean distance, 3.6 nm, can in turn be used to determine tDA=0.29 ns. Radius (R) of the micelles was determined by TEM (FIGS. 13A-13D).

FIGS. 15A-15F illustrate the response of mixtures of M1 and M2 to MMPs. FIG. 15A shows the fluorescence spectra of M1 and M2 (0.5 μM each with respect to PPA) with and without MMP-9 (10 nM) at times indicated following enzyme addition; λex=470. FIGS. 15B-15D show fluorescence intensity vs time plots via plate reader analysis, to monitor rearrangement of PPA-1 and PPA-2 into new FRET active aggregates; λex=490 and λem=590 nm. FIG. 15B shows detection of MMP-9 down to 10 pM of enzyme with M1 and M2 (at 0.5 μM, [PPA]). FIG. 15C shows detection of MMP-9 at 10 nM with varying concentrations of M1 and M2 shown with respect to [PPA], detectable down to 20 nM of polymer. FIG. 15D shows detection of cell-secreted (WPE1-NA45 cells) MMP-2 and -9 with varying concentrations of M1 and M2 shown with respect to [PPA]. Cells were seeded at 1.6×10⁴ cells/well in a clear bottom 96-well plate in DMEM. After 24 hrs, cell medium was added to solutions of M1 and M2. MMP-2 and -9 were at 0.048 nM and 0.005 nM respectively as quantified by an ELISA assay. Control was the non-MMP expressing MCF-7 cell-line cultured in the same manner. All reactions run in PBS, unless otherwise noted. FIGS. 15E-15F show TEM of M1 and M2 before (FIG. 15E), and after 24 hrs (FIG. 15F) following MMP-9 treatment.

FIG. 16 depicts a table of detection of MMP-9 at 10 mM in blood serum doped DMEM, a cell growth medium Detection of MMP in blood serum doped DMEM media via significant shortening in fluorescence lifetime indicating particle fusion upon cleavage of PPAs within M1 and M2.

FIG. 17A illustrates monomer synthesis of tert-butyl-(2-((2S)-bicyclo[2.2.1]hept-5-ene-2-carboxamido)ethyl)carbamate. FIG. 17B illustrates polymer synthesis.

FIG. 18 illustrates a SEC-MALS intensity plot of initially prepared 1₂₁-b-2₆-b-3₃ (blue) and following conjugation with Peptide 1 (green). SEC-MALS: 1₂₁-b-2₆-b-3₃; Mn=7459 g/mol, PDI=1.053. Peptide conjugate of 1₂₁-b-2₆-b-3₃; Mn=15270 g/mol, PDI=1.164.

FIG. 19 depicts a table of polymers (peptide substrate SEQ ID NOS:4 and 5), PPAs and resulting micelles.

FIG. 20A shows a graph of product vs time for reactions of M4 with MMP-9 at various substrate concentrations. FIG. 20B shows a graph of Initial rate vs substrate concentration. a indicates the literature value of Kcat/KM for standard peptide with MMP-9.

FIG. 21A illustrates MMP-9 cleavage of M1 and Peptide-1. Other trace is intact Peptide-1 without treatment with MMP-9. FIGS. 21B-21C illustrate MALDI-TOF mass spectra. MALDI-MS of Peptide-1 fragment (FIG. 21B) and fragment from M1 (FIG. 21C) cleaved by MMP-9. Mass calcd: 1398.6, Obs: 1399.7 (FIG. 21B) and 1399.2 (FIG. 21C).

FIG. 22 shows micelle counting via 20 nm Au NPs calibration visualized by TEM. 20 μL of M3 was mixed with 20 μL of 20 nm Au NPs at concentration of 7×10¹⁴ particles/L. 1243 M3 and 158 Au NPs were counted. TEM images shown here are representative of M3 mixed with Au NPs. M3 was counted as 5.51×10¹⁵ particles/L after calibration by Au NPs. Arrows indicate some representative 20 nm Au NPs visible clearly from TEM images as solid spheres, as opposed to open circles for the organic matter stained by uranyl acetate.

FIG. 23A shows a table of the weight average molar mass and aggregation number of M1, M2 and M3 from SLS. FIG. 23B shows a graph of the volume % vs the hydrodynamic diameter of the aggregates. It illustrates DLS of M1/M2 micelle mixtures mixed with non-activated MMP-9 (left) or activated MMP-9 (right).

FIGS. 24A-24B show DLS (FIG. 24A) and TEM (FIG. 24B) data of M3 mixed with MMP-9.

FIGS. 25A-25C show results of PPA injections into nude mice expressing HT1080 tumors. FIG. 25A shows a diagram of PPA-mediated aggregate formation. FIG. 25B shows the expression of the nanoparticles in the mice. FIG. 25C shows fluorescence of the nanoparticles in tumors.

FIG. 26 shows embodiments of a DNA-programmed micelle design. A copolymer 1₃₈-b-2₁₈ is conjugated with a DNA moiety, such as DNA-1 (SEQ ID NO:9), DNA-2 (SEQ ID NO:10), and DNA-3 (SEQ ID NO:11). The DNA-polymer can include other moieties such as PEG or F. The DNA-polymer undergoes cleavage and forms a micelle. The micelle comprises a DNA shell and a phenyl core.

FIGS. 27A-27D show analysis of the DNA-programmed micelles. FIG. 27A shows SLS analysis and FIGS. 27B, 27D show AFM analysis. FIG. 27C shows a TEM image of the micelles.

FIGS. 28A-28J show another example of DNA-programmed nanomaterial. FIG. 28A shows the DNA sequences (SEQ ID NOS:12 and 13). FIG. 28B shows the reaction used to form micelles and aggregates with DNAS based nanomaterial. FIGS. 28C-28D show images of the micelles (FIGS. 28C-28F) and aggregates (FIGS. 28G-28J).

FIGS. 29A-29C show another example of DNA-programmed nanomaterial. FIG. 29A: DNA, of the DPA is labeled with a donor fluorophore (fluorescein) and an acceptor fluorophore (rhodamine) that are positioned to undergo FRET. The resulting micelle is incubated with DNAₜ that is complementary to DNAₛ. A nicking enzyme (e.g., Nt. CviPII) that specifically cleaves the annealed DNA strand releases the acceptor fluorophore, which allows the donor fluorophore to emit fluorescence. FIG. 29B: Exemplary particle. FIG. 29C: Relative fluorescence as a function of time.

FIG. 30 illustrates that programmed amphiphilicity of the synthetic polymeric nanoparticles can enable reversible morphology changes. Altering the hydrophilic DNA-brush and/or hydrophobic particle core can allow a nanoparticle to change from a sphere to a cylinder (e.g., fiber) upon DNA cleavage by a nuclease (i). A nanoparticle that is a cylinder can change shape to be a sphere upon DNA annealing (ii) and change to the hydrophilic DNA-brush.

FIG. 31 illustrates an increase in fluorescence of fluorescent DNA-polymer micelles (substrate) in the presence of enzyme (DNAzyme, SEQ ID NO:14).

FIG. 32 illustrates fluorescence in the DNA-polymer nanoparticles during morphological changes induces by DNAzymes (SEQ ID NOS:15 and 16) and the DNA sequence in the particle shell (SEQ ID NOS:17 and 18).

FIG. 33 shows that the length of the nano fibers can be controlled by the nanomaterial.

FIG. 34 shows a diagram of switching the morphology of polymeric nanoparticles.

FIGS. 35A-35F show the conformational differences between spherical and cylindrical nanoparticles. FIG. 35F shows a schematic diagram of the DNA-polymer nanomaterial. Using J77 murine macrophage cells, we add either 0.1 nmole rhodamine and fluorescein co-labeled DNA-polymer nanofibers (FIGS. 35A-35D) or nanospheres (FIG. 35E) to the cells. Then, we added 0.1 nmole of complementary DNA from another 1 hr (FIG. 35B, 2 hrs (FIG. 35C) and 4 hrs (FIG. 35D).

FIGS. 36A-36B show fluorescence detected in mice injected with rhodamine and fluorescein labeled spherical and fiber nanomaterial structures. FIG. 36A shows fluorescence of the fibers. FIG. 36B compares the fluorescence of the spheres alone, the fibrils alone and the fibrils incubated with complementary DNA.

FIG. 37A shows the fluorescence detected in organs from mice injected with rhodamine and fluorescein labeled spherical and fiber nanomaterial structures. FIG. 37B shows the fibers present in serum 24 hours post-injection.

### DETAILED DESCRIPTION OF THE INVENTION

### I. Introduction

Provided herein are polymeric imaging agents that self-assemble in the presence of enzymatic activity associated with tumors or other disease states. Further provided methods for in vivo detection of cancer-associated or disease-associated enzymes based on imaging the polymeric nanostructures or nanoparticles thus formed. Described herein are methods for enzymatically triggered assembly of polymeric nanostructures for detection of cancer-associated enzymes in vivo. By detecting enzymatic signals associated with disease, one can sensitively determine the site, and extent of disease within a patient.

The formation of the nanoparticles indicates that enzymes are present and the aggregates can be detected using MRI or fluorescence techniques. According to the presently disclosed methods, whereas the polymer probes circulate in the bloodstream and diffuse through the interstices of normal and tumor tissues, the polymeric nanostructures formed by enzymatically-triggered assembly are too large to traverse the pores between endothelial cells and are hence trapped within the tumor tissue, leading to accumulation over time. This provides a unique mechanism to amplify the disease-associated enzymatic signal. By detecting such signals, one can sensitively determine the site and extent of disease within a patient.

Thus, provided herein are methods for detecting enzymatic activity through the accumulation of a nanoparticle structure upon reaction of a specially designed polymer with an enzyme. The formation of the particles constitutes the signal that enzymes are present. The signal is in the form of a detectable signal such as an MRI-signal and/or a fluorescent signal specific to the aggregate. This mode of detection allows an amplification of the disease-associated signal by way of the aggregation of an assembled particle within particular tissue. This also confers to a difference in the rate of clearance (and overall pharmacokinetics) of the material in the diseased tissue versus the systemic blood circulation. The outcome is an enhancement of signal to noise ratio. In addition, the particles are specific combinations of subunit polymers, giving amplified and specific signals depending on whether they are assembled or not.

### II. Definitions

The terms “nanomaterial,” “nanoparticle” refers to a material with morphological features on the nanoscale.

The term “probe” refers to a material, chemical, or biomolecule that is able to be used for detection and/or targeting of a biomolecular, biochemical and biophysical state, activity or moiety.

The terms “visualizable label,” “targeting agent” or “labeling agent” refers to a dye, quencher, reporter, chemical or molecule that is added to a polymer, polypeptide, nucleic acid, chemical or molecule. In some instances, the “visualizable label,” or “labeling agent” is detectable.

The terms “self-assembly” and variations thereof refer to a process in which a components adopt and form an organized structure as a consequence of specific, local interactions among the components themselves, without external direction or interactions.

The terms “polypeptide” and “peptide” refers to amino acids linked by peptide bonds.

The term “DNAyme” refers to an enzyme that cleaves DNA. Non-limiting examples include an endonuclease, an exonuclease or a DNA nicking enzyme.

The term “micelle” refers an aggregate, assembly, or arrangement of molecules with a hydrophilic region in contact with the surrounding solution and a sequestered nonpolar (lipophilic or hydrophobic) region.

The term “cryptic amphiphile” refers to a hydrophilic polymer that switches to an amphiphilic polymer and assembles via aggregation in response to stimuli. Stimuli include a molecule, chemical, polypeptide or nucleic acid that can induce a modification in conformation, molecular structure, chemical structure, function, activity, or other characteristic properties.

The term “terminating agent” refers to a chemical moiety that is linked to a polymer. In some instances, the terminating agent is conjugatable such as PFP and carboxylic acid. In other instances, the agent is hydrophilic such as PEG. In yet other instances, the terminating agent is a dye, quencher or visualizable label. Non-limiting examples of a dye or a quencher are Boc-amine, thioacetate, fluorescein, rhodamine, coumarin, and Dabcyl. In other instances, the terminating agent is a MRI contrast agent such as a Gd³⁺ reporter.

The term “MMP-associated cancer” refers to a cancer characterized by an overexpression of a matrix metalloproteinase, such MMP-1, -2, -7, -9, -12, -13, -14, and -15, or a related gene. A MMP-associated cancer includes various human cancers such as, but not limited to breast cancer, cervical cancer, prostate cancer, colitis cancer, lung cancer, colon cancer, bladder cancer,

The terms “subject” or “organism” refer to a human or an animal.

As used herein, the following terms have the meanings ascribed to them unless specified otherwise.

### III. Detailed Descriptions of Embodiments

Provided herein are methods of detecting a cancerous tissue in a subject comprising: (a) obtaining a hydrophilic polymer probe comprising: a hydrophilic polymer containing an MMP cleavable polypeptide, and a visualizable label; (b) administering a hydrophilic polymer probe to the tissue in an amount sufficient to provide a detectable image; (c) allowing cleavage of a hydrophilic polymer probe by a cancer-associated enzyme, wherein the cleaved hydrophilic polymer probe self-assembles into an amphiphilic polymer aggregate, comprising of amphiphilic polymer and a visualizable label, at the cellular location to be imaged; and (d) imaging the tissue, wherein a detectable image of the self-assembled particle aggregate in the tissue indicates the presence of cancerous tissue or a tumor.

In some embodiments, the cancer-associated enzyme is a protease, a metalloprotease, or a matrix metalloproteinase. In exemplary embodiments, the cancer-associated enzyme is MMP-2 or MMP-9. In some embodiments, the cancer is a MMP-mediated malignancy, sarcoma or metastasis.

In some embodiments, the visualizable label is selected from a group consisting of fluorophores, Gd³⁺ reporters or combinations thereof. In some embodiments, the amphiphilic polymer aggregate forms a micelle.

Further provided are methods of detecting a diseased tissue in a subject comprising: (a) obtaining a hydrophilic polymer probe comprising: a hydrophilic polymer containing a cleavable DNA fragment, and a visualizable label; (b) administering a hydrophilic polymer probe to the tissue in an amount sufficient to provide a detectable image; (c) allowing cleavage of a hydrophilic polymer probe by a nuclease, wherein the cleaved hydrophilic polymer probe self-assembles into an amphiphilic polymer aggregate, comprising of amphiphilic polymer and a visualizable label, at the cellular location to be imaged; and (d) imaging the tissue, wherein a detectable image of the self-assembled particle aggregate in the tissue indicates the presence of diseased tissue.

In some embodiments, the DNA fragment is a DNA sequence comprising a sequence that can be cleaved by a nuclease or an enzyme that recognizes specific DNA sequences. In some embodiments, the nuclease is an endonuclease, an exonuclease, a DNA nicking enzyme, or a enzyme that can cleave DNA.

In some embodiments, the diseased tissue is a cancerous tissue.

In some embodiments, the self-assembled particle aggregate comprises polypeptide and DNA sequences.

Further provided are methods of treating a subject having cancer, the method comprising: (a) obtaining a hydrophilic polymer probe comprising: a hydrophilic polymer containing an MMP cleavable polypeptide, targeting polypeptide, and a therapeutic agent; (b) administering a hydrophilic polymer probe to the tissue in an amount sufficient to provide a therapeutic dosage to the subject; (c) allowing the localization of a hydrophilic polymer probe to the targeted tissue in the subject as directed by the targeting polypeptide; (d) allowing cleavage of a hydrophilic polymer probe by cancer-associated enzyme, wherein the cleaved hydrophilic polymer probe self-assembles into an amphiphilic polymer aggregate, comprising of amphiphilic polymer and a therapeutic agent, at the cellular location to be treated, thereby treating a subject with cancer.

In some embodiments, the therapeutic agent is selected from the group comprising doxorubicin, paclitaxel, cisplatin, tyrosine kinase inhibitors, topoisomerase inhibitors, alkylating agents, antimetabolites, anthracyclines, antitumor agents, and combinations thereof.

Also described herein is an enzymatically-directed self-assembled amphiphilic polymer aggregate comprising: an amphiphilic polymer, which comprises of a hydrophilic cryptic-amphiphile with a cleaved MMP-specific polypeptide, and a labeling group.

Further provided is an enzymatically-directed self-assembled amphiphilic polymer aggregate comprising: an amphiphilic polymer, which comprises of a hydrophilic cryptic-amphiphile with a cleaved DNA fragment and a labeling group.

Further provided is a hydrophilic polymer probe, which is prepared by a preparation method comprising synthesizing a polymer using the method of ring-opening metathesis polymerization, and incorporating labeling groups, therapeutic agents and/or targeting groups into said synthesized polymer.

In some embodiments, hydrophilic cryptic-amphiphile is generated from a a norbornene derivative or a norbornene-based monomer.

Further provided are methods of treating a MMP-mediated cancerous tissue of a subject's body, comprising providing a therapeutic agent conjugated to hydrphophilic polymer probe in a manner to direct self-assembly of the hydrophilic polymer into amphiphilic polymer aggregates and delivery of the drug to the MMP-mediated cancerous tissue.

In some embodiments, provided are methods of treating a diseased tissue of a subject's body, comprising providing a therapeutic agent conjugated to hydrphophilic polymer probe in a manner to direct self-assembly of the hydrophilic polymer into amphiphilic polymer aggregates and delivery of the drug to the diseased tissue.

In some embodiments, provided are methods of imaging a MMP-mediated cancerous tissue of a subject's body, comprising providing a labeling agent conjugated to hydrophophilic polymer probe in a manner to direct self-assembly of the hydrophilic polymer into amphiphilic polymer aggregates and delivery of the labeling agent to the MMP-mediated cancerous tissue.

In some embodiments, provided are methods of imaging a diseased tissue of a subject's body, comprising providing a labeling agent conjugated to hydrophophilic polymer probe in a manner to direct self-assembly of the hydrophilic polymer into amphiphilic polymer aggregates and delivery of the labeling agent to the diseased tissue.

In some embodiments, the diseased tissue is a cancerous tissue.

In some embodiments, the hydrophilic polymer is generated using a peptide monomer. In some embodiments, the peptide monomer comprises a peptide. In some instances, the peptide sequence is G-PEG₂Suc-GPLGLAL-PEG₂Suc-NH₂ (SEQ ID NO:1), GPLGLAGK(cNAc)-PEG₂Suc-NH₂ (SEQ ID NO:2) or GPLG-NH₂ (SEQ ID NO:3). In some embodiments, the hydrophilic polymer is formed by direct polymerization of the peptide.

In some embodiments, the peptide monomer is a norbornene derivative or a norbornene-based monomer.

In certain embodiments, an assembly of processed cryptic-amphiphiles into nanoparticles can be used in subjects and in vitro through FRET lifetime imaging. In other embodiments, the localization of assembled nanostructures can be characterized both macroscopically with in vivo FRET fluorescence lifetime imaging (FRET-FLIM) and histologically with fluorescence microscopy. Described herein is the use of optimal fluorescence lifetime probes for FRET-FLIM which is of general interest and utility in the field of in vivo molecular imaging.

In certain embodiments, provided are polymeric MRI contrast agents that self-assemble selectively into nanoparticles in situ for tumor detection. Cryptic-amphiphiles give rise to a detectable MRI signal via enzyme-directed assembly. In some embodiments, cryptic-amphiphiles are enzymatically processed by tumor associated enzymes in vitro leading to amphiphile formation and assembly into nanoparticles with increased relaxivity. In other embodiments, once processed, the amphiphiles assemble into nanoparticles selectively in tumor tissue and provide a specific, robust MRI signal over background in mouse tumor models. In yet other embodiments, the materials will only be assembled into nanoparticles inside tumor tissue.

Further provided are nanoparticles that home to tumor tissue and once there undergo a dramatic morphology change in response to tumor-specific enzymes to enable site-specific accumulation. In some embodiments, nanoparticles bearing targeting moieties home to tumor tissue selectively. In some embodiments, enriched disease-associated enzymes direct morphology change of appropriately designed nanoparticles in situ. In some embodiments, nanoparticle morphology change leads to small molecule release and imaging agent activation in tumor tissue, thus yielding a tumor-specific diagnostic nanoparticle drug reservoir.

Further provided are methods to visualize the probe, Fluorescence studies in vitro and in vivo were performed for design optimization and analysis. Polymers decorated with Gd³⁺-chelate enable the probe to be visualized by MRI imaging. The MRI-based method yields clinically useful positive contrast combined with excellent spatial resolution. Pathological biomolecules are present in concentrations far below this detection limit, making amplification a prerequisite to MRI-based molecular imaging of disease biomarkers. In some embodiments, the presently described polymers conjugated with Gd³⁺ reporters amplify the signal from biomolecules, and are detected on MRI-based imaging systems.

A number of polymerization methods can be applied to the formation of highly functionalized polymers. In some embodiments, ring-opening metathesis polymerization (ROMP) because of ease of implementation, high functional group tolerance (allowing high density incorporation of dyes and drugs by direct polymerization), and its living. Furthermore, ROMP allows the synthesis of polymers with given terminal chemistry (R′ group via termination agent incorporation), and block copolymer structure (R- and R″-) via norbornene monomers modified appropriately. The polymer backbone has been shown to be bioorthogonal and inert in studies by others in the art. In some embodiments, reporters, targeting groups, and drugs are incorporated into the amphiphilic polymers through backbone incorporation via modified norbornene monomers (R- and R″-), by termination group chemistry (R′-) or by modification of the R-, or R″-groups post-polymerization. For instance, solid-phase peptide synthesis with modified amino acids is used to install fluorophores in the peptide-side chains on solid support and then conjugated to one of the blocks via amide coupling chemistry. In other embodiment, the polymer backbone comprises non-biodegradable all side chains linked via degradable linkers (e.g. esters) giving an ultimately clearable, low molecular weight skeleton for clearance post accumulation. In some embodiments, highly functionalizable metal-free biodegradable polymer frameworks are used.

In some embodiments, the polymeric fluorescent imaging agents described herein self-assemble selectively into nanoparticles in situ for tumor detection. In some embodiments, fluorescence based methodologies are used in the development, optimization, and understanding of cancer-associated stimuli induced assembly of the cryptic-amphiphile probe system and assess its potential at a level of detailed molecular assembly, for Enzyme-directed Assembly of Particle Theranostics (EDAPT).

In some embodiments, the assembly of processed cryptic-amphiphiles into nanoparticles described herein were detectable in live organisms and in vitro through FRET lifetime imaging. In some embodiments, ring-opening metathesis polymerization (ROMP) is used to generate a cryptic-amphiphile such as a Type 1 cryptic-amphiphile and a Type 2 cryptic-amphiphile. In some instances, a critical micelle concentration (CMC) determines the amphiphile architecture. Investigations on related materials suggest nanomolar (nM) CMCs. In some embodiments, aggregation kinetics are monitored by FRET to determine what types of morphologies and sizes of assemblies are formed. In other embodiments, enzyme kinetics are measured with respect to polymeric peptide substrates utilizing cryptic-amphiphile Type 2.

In some embodiments, FRET-FLIM is used to provide a means for analysis of the assembly process in conjunction with detailed materials characterization methods including transmission electron microscopy (TEM), atomic force microscopy (AFM), scanning electron microscopy (SEM), dynamic light scattering (DLS), and confocal fluorescence microscopy. In some instances, peptides are synthesized containing dye-quencher pairs as well as polyethylene glycol moieties directly incorporated during solid phase synthesis and confirmed to be substrates for MMP cleavage. These peptide sequences form the peptide brush incorporated by post-synthetic modification into cryptic-amphiphiles. In studies, such conjugation reactions generated low polydispersity peptide-copolymers that are readily characterized by SEC-MALS. Direct polymerization of peptide-modified norbornene monomers by ROMP was also performed. This extraordinary level of functional group tolerance allows for consistent control over the number of peptides incorporated.

In other embodiments, FRET pairs of long wavelength (near-IR, Cy5.5 and Cy7) dyes are incorporated to track polymer assembly and enzyme kinetics in vitro and in vivo. A polymer was synthesized with a Cy7 terminus and an otherwise identical polymer was synthesized, end-terminally labeled with Cy5.5. When mixed together and treated with MMPs, micelles showed indicative fluorescence lifetimes because of the FRET pairs present in the same aggregate.

Mixed dye systems allow for tracking of intact aggregates in vivo via whole organism fluorescence lifetime imaging (FLIM). In some embodiment, multiple dye pairs are incorporated into the polymer nanomaterials. Non-limiting examples of dye pairs include Rhodamine/Fluorescein, EDANS/DABCYL, Fluorescein/Dabcyl, Cy 7/Cy 5.5, and near-IR dye pairs. In some embodiment, the labeling shows the aggregate morphology transition concurrent with cancer tissue labeling. For instance, “red” and “green” labeled polymers generate mixed populations of micelles with specific FRET properties that arevisualized by whole organism imaging and via microscopy following organ removal and analysis.

Typically, a certain concentration of polymer should be present to undergo aggregation and hence, accumulation. This relates directly to the CMC of the materials following enzymatic cleavage to generate amphiphiles in situ. In some embodiments, CMCs on the order of nanomolar concentrations (nM) are sufficient based on projected injectable quantities in the micromolar range. In other embodiments, it is necessary to incorporate additional targeting moieties to drive the equilibrium to promote aggregation and accumulation. In certain embodiments, the polymers are incorporated with peptide-based (cyclic-RGD) targeting moieties. In other embodiments, the polymers are bound in a modular fashion to small molecule targeting groups such as but not limited to, folic acid.

In some embodiments, the modular polymer design described herein is manipulated to optimize structural characteristics of the peptide-copolymer systems. In certain embodiment, the modular polymer design is modified to affect proteolysis rates via specific and non-specific protease action. For instance, parameters such as the size of the PEG-moeities incorporated into the shell are modulated to improve the structural characteristics. In addition to targeting groups, cross-linking groups are utilized to improve or direct accumulation. In some instances, the polymer based micellar particles are crosslinked in their shell regions to form covalently stabilized particles that no longer dissociate at concentrations lower than the CMC. Crosslinking aids in stabilization upon aggregation in vivo. In other embodiments, the crosslinking strategy employs bioorthogonal reactive groups that selectively lead to crosslinking of amphiphiles at sufficient rates only when the two mutually reactive groups are in high concentrations and not when they are in low concentrations (i.e. kinetic control). For instance, the crosslinking strategy such as, but not limited to, the Bertozzi's copper free “click” reaction is utilized. In other instances, the crosslinking strategy increases the reaction rate of the requisite azide and the cyclooctyne, culminating in the ability to carry out this reaction in a living organism. For instance, copper-free click chemistry function sin a polymer setting and is to crosslink particle shells. In some embodiments, the polymer crosslinking reaction utilizes a copper free system and/or cyclooctyne-azide system that exhibits slow kinetics at low concentrations, such that no crosslinking occurs when in the dilute cryptic-amphiphile state. It should be noted that once enzymatically processed and assembled into particles, crosslinking occurs due to the induced close spatial proximity of the amphiphiles, giving rise to substantially higher effective concentrations of the mutually reactive groups. In other embodiments, azides and cyclooctynes in the hydrophilic block are added to the cryptic-amphiphiles.

In some embodiments, in vivo whole organism studies can be performed to show that in vitro optimized cryptic-amphiphiles bearing appropriate fluorophores for FRET-FLIM are properly assembled into particles in response to enzymatic conversion into amphiphiles. In certain embodiment, the nanomaterial probes are administered to live mice bearing tumor models to demonstrate in vivo efficacy. Several animal models of human tumors containing overactive MMPs are known, such as the HT-1080 xenograft model and the MMTV-PyMT transgenic breast cancer model. The HT-1080 xenograft model employs human fibrosarcoma cells and contains highly active human MMPs. The MMTV-PyMT model has been shown, through exhaustive molecular and histological characterization, to be a robust surrogate for human breast cancer. This model is based on the specific expression of PyMT (polyoma virus middle T oncoprotein) in mammary epithelium, which leads to de novo carcinoma formation in a substantial fraction of mice as well as metastatic spread. In one embodiment, the two mouse models are injected intravenously with cryptic-amphiphiles as well as control versions of cryptic-amphiphiles that are identical except that the peptide sequence will be synthesized to contain D-amino acids instead of L-amino acids. This stereochemical “mutation” is an ideal control because the size, charge, and other physicochemical properties of the peptide are completely conserved between the two, but the D-amino acid control is far more resistant to proteolysis by MMPs. Each mouse is placed in the fluorescence lifetime imaging (FLIM) device, where a baseline image is recorded, followed by injection and subsequent re-imaging in the relevant fluorescence channels. Uptake of the probe into tumors is compared to background organs within each mouse as an internal control. In addition, specific uptake into tumors in mice injected with L-amino acid containing cryptic-amphiphiles is compared to uptake of D-amino acid containing control analogues. Specific uptake and kinetics are determined by repetitive imaging. Once the timepoint(s) for optimal accumulation of probe over background and over control is determined, mice are again injected with experimental and control probes, allowed to live for the predetermined optimal amount of time, then sacrificed. Tumors and organs are collected and analyzed by fluorescence histology. This allows for the determination on the cellular level of where probes have accumulated.

In some embodiments, the localization of assembled nanostructures are characterized both macroscopically with in vivo FRET fluorescence lifetime imaging (FRET-FLIM) and histologically with fluorescence microscopy. Conventional in vivo imaging methods are incapable of distinguishing between cryptic-amphiphiles and assembled particles due to the mm spatial resolution. FRET, which occurs over short distance (order of nanometers) between donor and acceptor, overcomes this limitation. FRET causes donor fluorescence intensity and lifetime to be quenched. By synthesizing the polymer to position the donor and acceptor at specific proximities for the different configurations (e.g. cryptic-amphiphiles vs assembled nanoparticle), there will be specific FRET-efficiency and corresponding lifetimes. In one configuration, the donor and acceptor is separate, as water soluble, individual, non-aggregated cryptic-amphiphiles, such that the donor fluorescence lifetime is not quenched. In another configuration (e.g. assembled nanoparticle) the dyes are in close proximity such that the donor fluorescence lifetime is quenched.

In some embodiment, a simple fluorescence probe comprises fluorescein (donor fluorophore) and Dabcyl (dark quencher) as a FRET pair with an enzyme-cleavable DNA linker. Studies shows that the fluorescence lifetime clearly increases from the uncleaved probe ˜2.2 ns to the cleaved probe ˜3.2 ns. The data demonstrates that the quenching is dynamic and is due to FRET. The results also shows that an operating dye/quencher pair givesrise to a detectable lifetime switch.

In certain embodiments, polymeric MRI contrast agents self-assemble selectively into nanoparticles in situ for tumor detection. In some embodiments, accumulation of MRI-contrast agent in one particular location gives rise to enhancement of contrast in that location by concentrating agent, and/or possibly by an enhancement of relaxivity (r₁) of the agent itself via incorporation into a larger, slower tumbling aggregate. Cryptic-amphiphiles bearing Gd³⁺ are be enzymatically processed by MMP-2/9 in vitro leading to amphiphile formation and aggregation into nanoparticles with increased relaxivity. In some instances, cryptic-amphiphiles are prepared and labeled with Gd³⁺ via chelation to DTPA or DOTA groups polymerized directly into the polymer backbone. ROMP-active norbornene monomers and terminating agents containing DTPA and DOTA are prepared. In one embodiment, assays are performed for the determination of enzyme induced changes on relaxation parameters, such as NMR-based assays at various [Gd³⁺] and ICP-MS (inductively coupled plasma mass spectrometry). It is known to those in the art that, in principle, slowing of the rotational mobility of paramagnetic MRI contrast agents leads to increases in relaxivity (r₁) and hence higher signal on T1-weighted MRI, which is extremely useful for clinical imaging. In some embodiment, measurements of r₁ are determined empirically at clinically relevant field strengths before and after MMP-2/9 treatment in vitro.

Once processed, the resulting amphiphiles are assemble into nanoparticles selectively in tumor tissue and provide a specific, robust MRI signal over background in tumor models (e.g., mouse tumor model). In some instance, probe is injected into mouse tumor models to verify that the Gd³⁺ labeled probes exhibit the same accumulation kinetics as their fluorescent analogues. Efficacy experiments are undertaken using the probes and their D-amino acid analogue controls. MRI images are quantified and statistical analysis is performed to test if uptake of the probe is significantly higher than background and control. Additionally, mice are injected with probe, allowed to live for the predetermined optimal time at which specific accumulation is highest, and then sacrificed. Tumor and organ homogenates are analyzed by ICP-MS to determine, in a complementary fashion, the amount of Gd³⁺ and hence probe in the tumor compared to other tissues and organs as well as comparing tumors from mice injected with experimental probe vs mice injected with non-cleaved control (D-amino acid probe).

In certain embodiments, the nanoparticles home to tumor tissue and once there undergo a dramatic morphology change in response to tumor-specific enzymes to enable site-specific accumulation. A nanoparticle-based system is created to be carriers of internalized drugs. The changeable shape, size and morphology of a synthetic nano- or microscale vector directs its pharmacokinetics and targeting capabilities in vivo. In certain instances, a unimer polymer to amphiphilic micelle aggregate switch is guided by enzymes. In other instances, a well-defined, preformed 20-30 nm peptide-amphiphile nanoparticle micelle is generated so that it is capable of undergoing a dramatic change in morphology upon reaction with MMPs. This packaging system is capable of protecting and releasing drugs over time either as blood circulating reservoirs or actively targeted agents.

Controlling the PK and targeting of small molecule drugs and diagnostics is at the core of medicinal chemistry, pharmaceutical science and biomedical imaging. The intense interest in nanoscale vehicles designed for targeted delivery and detection in vivo is predicated on the idea that such materials may infer their PK, bioavailability and targeting properties on small molecules and other cargo including biomolecules.

Depending on the design and intended function of the nanoparticles, they respond in a number of ways such as degrading and releasing their payload, changing their morphology, or undergoing modifications that affect their aggregation state, thereby causing a change in the imaging properties of the material. Block copolymer amphiphiles are advantageous for use in functional, stimuli-responsive systems because changes in the chemical or physical nature of the amphiphile leads to formation, destruction, or morphological transformation of the supramolecular aggregates formed. In some embodiment, peptides, as enzyme substrates, are used as hydrophilic head groups in amphiphiles to generate enzymatically regulated materials. Four peptide-copolymer amphiphiles were synthesized and assembled into very well-defined spherical micelles (see, Example 2). ROMP-based peptide-polymeric materials formed ordered structures through self assembly. These materials underwent enzyme directed, dramatic increase in observed size in solution (DLS) via aggregation of the small particles through truncation of the peptide shell, to give large “network” structures (see, TEM/SEM images in Example 2). In some embodiments, nanomaterials with differential PK based on species adopt much larger structures in tumor tissue vs normal tissue.

In certain embodiments, nanoparticles bearing targeting moieties home to tumor tissue selectively. Particles accumulate in tumor tissue passively prior to enzymatic action, via the EPR (enhanced permeability and retention) effect. In some embodiment, targeting groups are incorporated at the terminus of the polymers via direct polymerization (norbornene-modified RGD), termination chemistry (alkene-modied RGD), or by post-polymerization modification. In some instances, fluorescently labeled particles (polymers synthesized with dyes directly through the backbone, at peptide sequences, and at termini as discussed herein) are monitored in vitro against human cancer cell lines for their ability to target to cell surfaces and be internalized. Analysis is conducted by FACS and microscopy. Especially interesting cell lines include HTB-14 that overexpress both integrins and MMPs allowing RGD and MMP based targeting to be analyzed.

In some embodiments, tumor-enriched enzymes direct morphology change of appropriately designed nanoparticles in situ. Decoration of the particles with dyes and MRI chelates allows tracking of the particles. In one embodiment, particles are synthesized with a particular dye on its surface, with another particle expressing the other member of the FRET-pair. FRET pairs will come together only upon enzyme-driven aggregation of particles. In another embodiment, Gd-DOTA and Gd-DTPA decorated particles are synthesized. Actively and passively targeted particles are assessed using animal models. In some embodiment, the method comprises utilizing a micellar nanoparticle system capable of carrying materials protected from solution within the hydrophobic core.

In some embodiments, morphology change leads to small molecule release and imaging agent activation in tumor tissue, thus yielding a tumor-specific theranostic nanoparticle drug reservoir. In some embodiments, noncovalently encapsulated drugs are release upon particle morphology change. Upon phase transition and reorganization of amphiphiles to larger aggregates, expulsion of their contents occurs. In some embodiments, covalently bound drugs are released via the action of esterases and/or upon exposure to physiological pH. Eventual degradation of the remainder of the peptide shell and biodegradable side chain linkages provides a source of drug release localized by the initial particle aggregation. In some embodiments, drug monomers (norbornene-Dox, norbornene-Etoposide) are synthesized and incorporated into the polymer framework. Efficacy studies are conducted on animal models utilizing materials as described herein for targeting studies in vivo and coupled with in vitro cell studies. Particles are loaded with doxorubicin or etoposide, both cytotoxic anticancer agents, linked to the polymer backbone by a cleavable, biodegradable linker. Doxorubicin is a clinically effective and standard drug for studies of this type. Etoposide is from the podophyllotoxin familyof cytotoxins with highly effective cytotoxic properties but suffering from significant off-target effects.

### EXAMPLES

### Example 1

### Enzyme-Responsive Micellar Nanoparticles

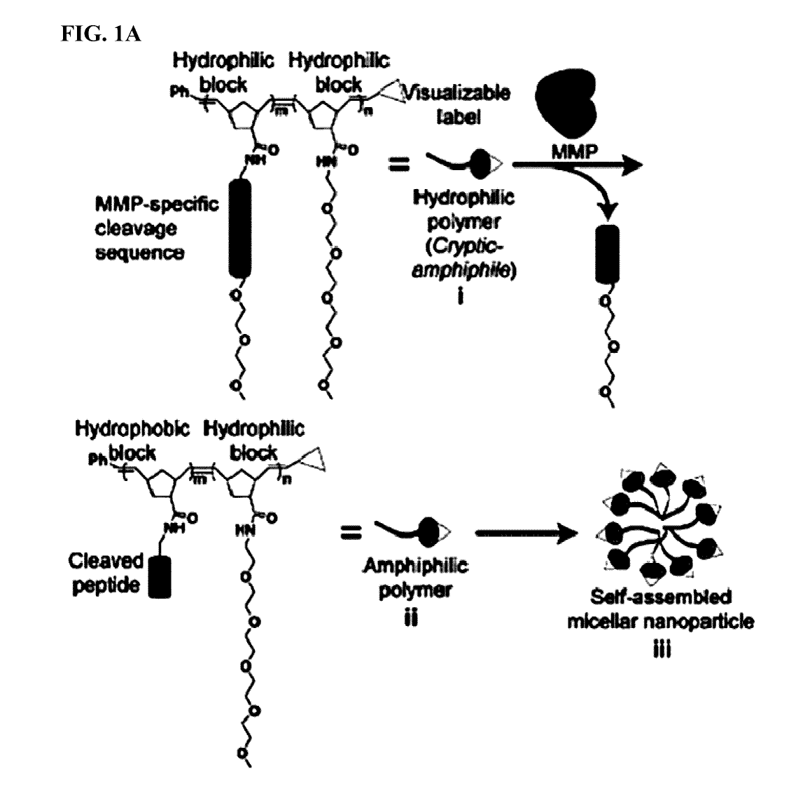

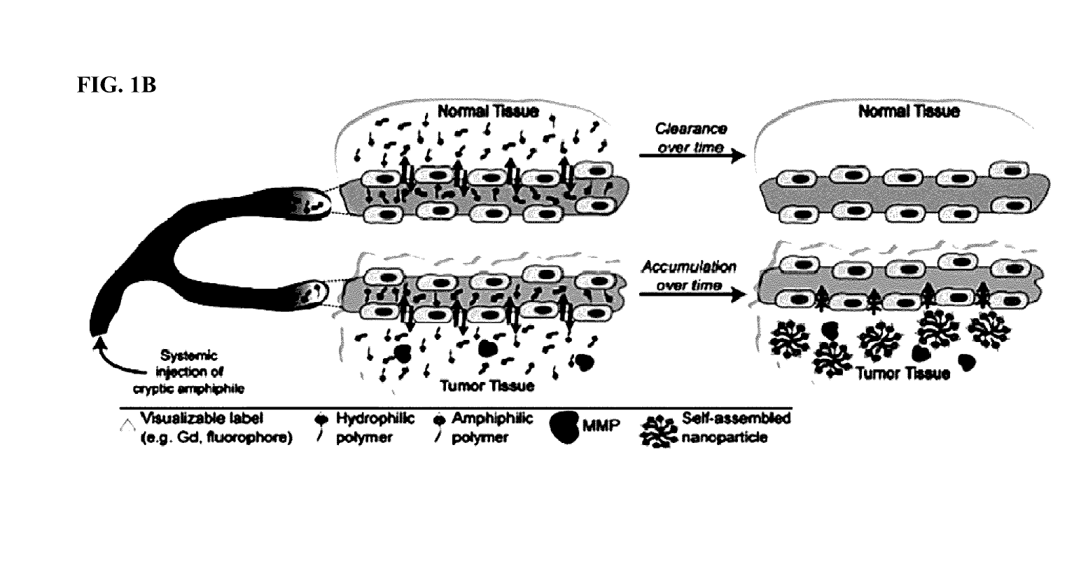

The example illustrates enzyme-responsive micellar nanoparticles. An example of a MMP-responsive peptide micellar nanoparticle is present in FIG. 1A. A hydrophilic polymer probe (i) is composed of a hydrophilic block containing a MMP cleavable peptide (dark blue), a hydrophilic PEG block, and a visualizable label (Gd³⁺/fluorophore). Upon encountering MMP, the peptide is cleaved, leaving behind a hydrophobic remnant. This constitutes a hydrophilic to amphiphilic switch as the polymer now contains a hydrophobic block and a hydrophilic block (ii). This amphilicity drives self-assembly to yield micellar nanostructures (iii), tunable in size on the order of tens to hundreds of nanometers in diameter. An in vivo example is presented in FIG. 1B. Following IV injection, intact polymer (i) circulates throughout the bloodstream and diffuses into and out of the interstitium of normal and tumor tissues due to its relatively small size (tunable in the range of <10-100 kDa). In tumor tissue, MMPs are upregulated and are poised to cleave their cognate peptide on the polymer, yielding a polymeric amphiphile, which then undergoes self-assembly to form nanostructures that are too large to traverse the pores between endothelial cells and are hence trapped within the tumor tissue, leading to accumulation over time. Probe that does not accumulate in tumors remains unimeric and will be cleared by renal filtration, giving rise to a high signal-to-noise ratio, to be detected by MRI and/or fluorescence. Fluorescence will be read out by lifetime imaging of FRET pairs. In the case of probe labeled with Gd³⁺, MRI-contrast is enhanced by local accumulation and a per-Gd³⁺ relaxivity increase due to slower tumbling rates upon nanostructure assembly. It should be noted that accumulated material can clear, albeit with slowed kinetics.

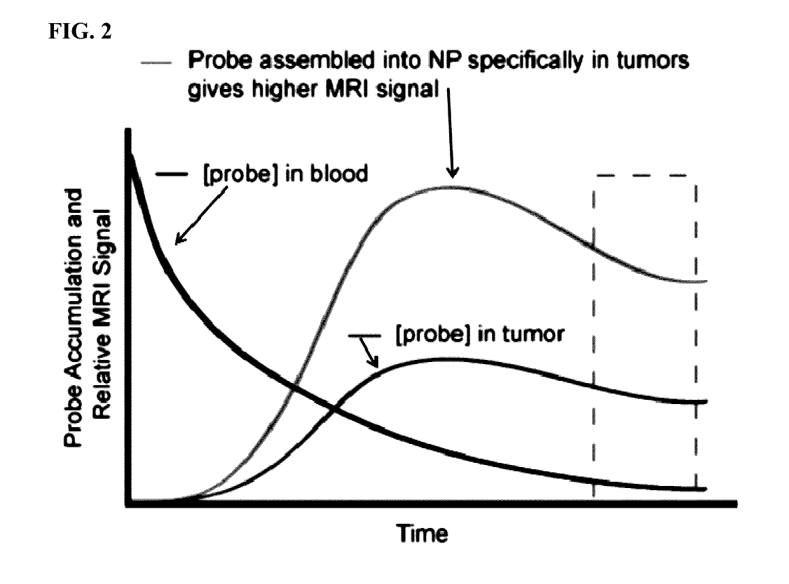

Enzyme-responsive micellar nanoparticles are designed to have optimal pharmacokinetic profiles and detecting profiles for tumors (FIG. 2). Upon injection of the nanomaterial, concentration is highest in blood, but diminishes over time due to somatic distribution and renal clearance (black curve). Once acted upon by intratumoral enzymes (e.g., MMPs), the nanomaterial accumulates specifically, leading to a higher concentration of a detection reporter (MRI reporter or fluorescent reporter) in tumors (red curve). Owing to its assembly into particles with intrinsically lower tumbling rates, the detection reporter increases and/or overall relaxivity of the particle is enhanced over unimer probe (green curve), yielding an even larger detectable difference between blood and tumor. Imaging in the time window defined by the box will lead to specific tumor detection. Slower clearance kinetics ensures window expansion without irreversible accumulation.

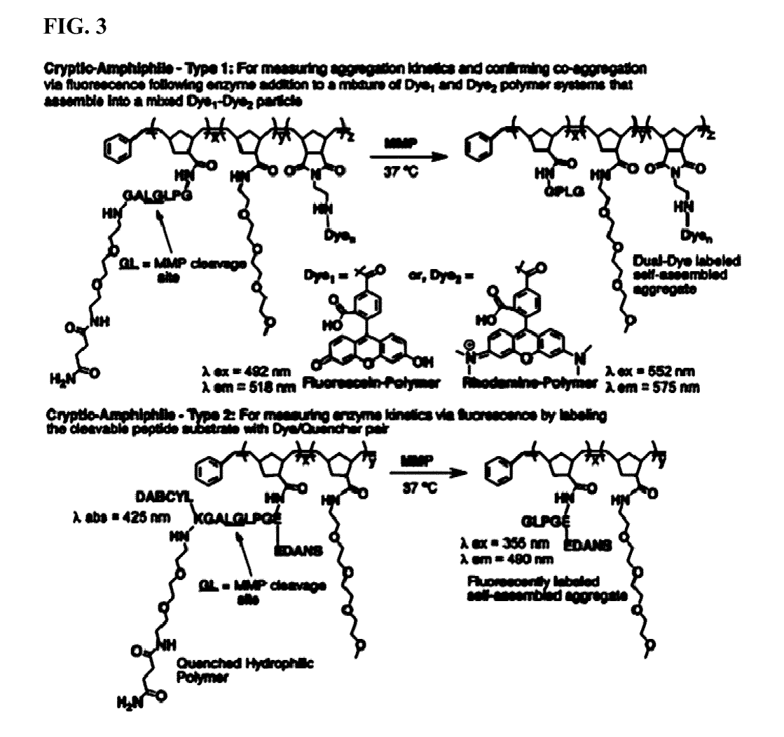

Enzyme-responsive fluorogenic peptide-programmed nanomaterials are synthesized by cryptic-amphiphile such as type 1 and type 2 (FIG. 3). For Type 1, we synthesized a variety of dye pairs modified for incorporation into ROMP-polymers via post-synthetic modification, end-termination chemistry (symmetrical alkenes and/or enol ethers, for cross-metathesis), or direct polymerization via norbornene-modifed monomers (as shown). Shown are Rhodamine/Fluorescein systems that show FRET-FLIM in our studies. Block sizes, x, y, z are easily varied to tune amphiphilicity of MMP product. Peptides are conjugated post-polymerization. For Type 2, the peptide is labeled at either side of an enzyme cut-site.

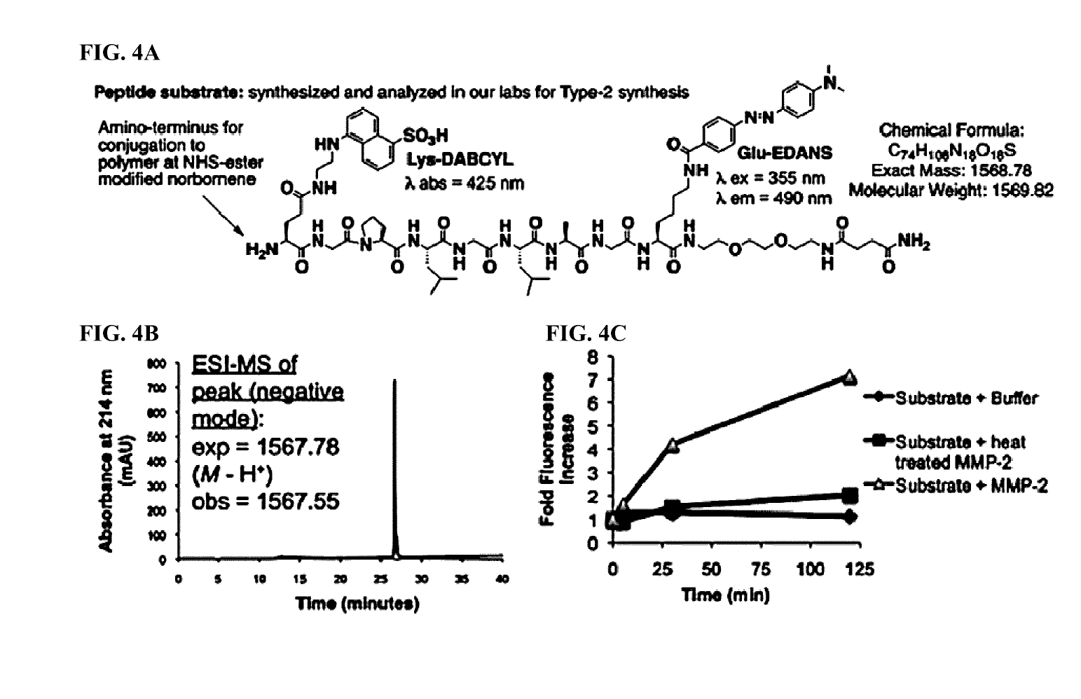

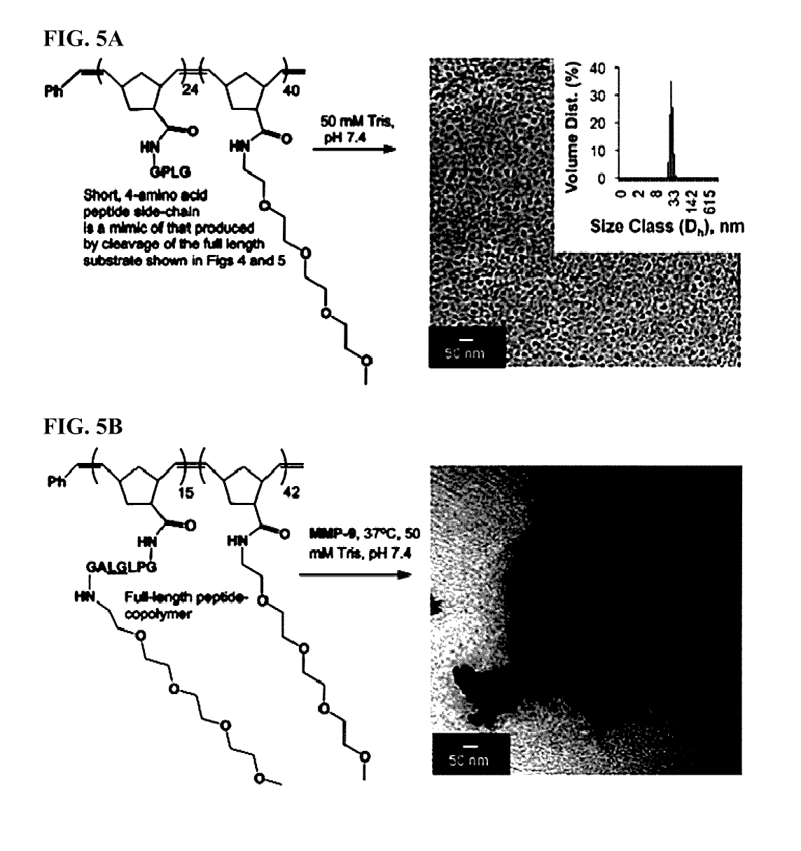

Synthesis of the peptide substrate is described in detail in Example 2. FIG. 4A shows a pure peptide, directly labeled fluorogenic substrate used for conjugation to a NHS-norborene polymer. The conjugation formed a Type 2 cryptic amphiphile. Fluorescence increase confirms performance as a substrate with active MMP-2 (FIG. 4C). Heat denatured enzyme control to test buffer influence, shows low activity.

Peptide programmed nanoparticles self-aggregated in response to enzyme cleavage of peptide substrate. Truncated peptide mimicking cleavage product assembled to give 30 nm particles shown by TEM and DLS (FIG. 5A). Cryptic-amphiphile assembled into amorphous aggregates upon treatment with MMP-9 (FIG. 5B). Cleavage occurs at Gly-Leu bond as underlined. No aggregation was seen in the absence of MMP-9.

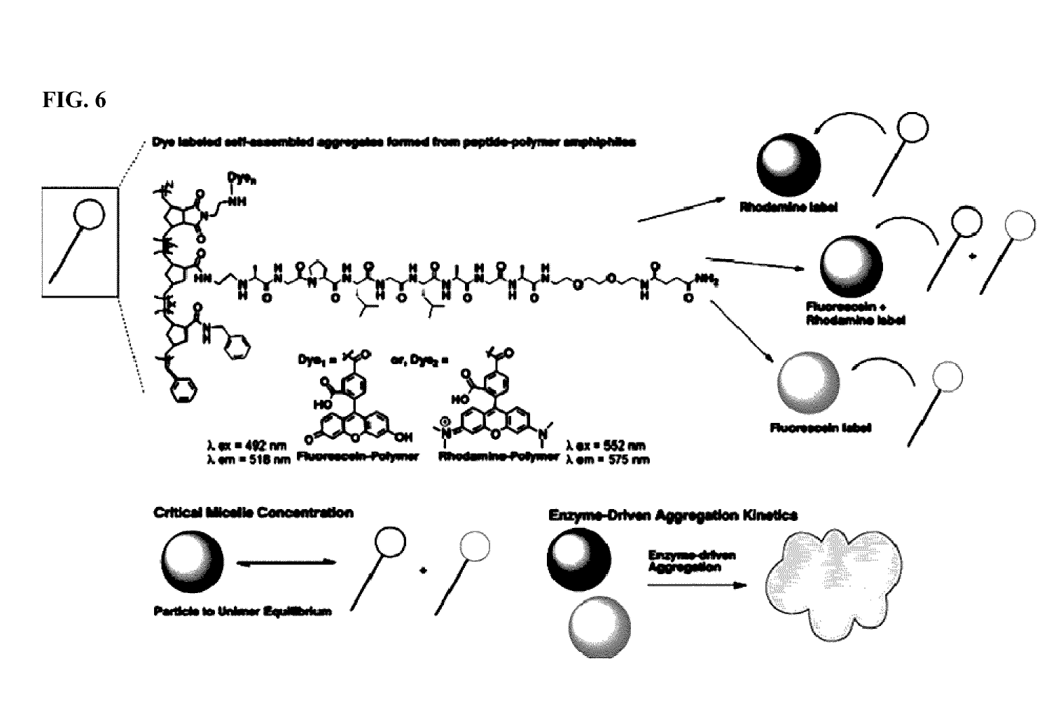

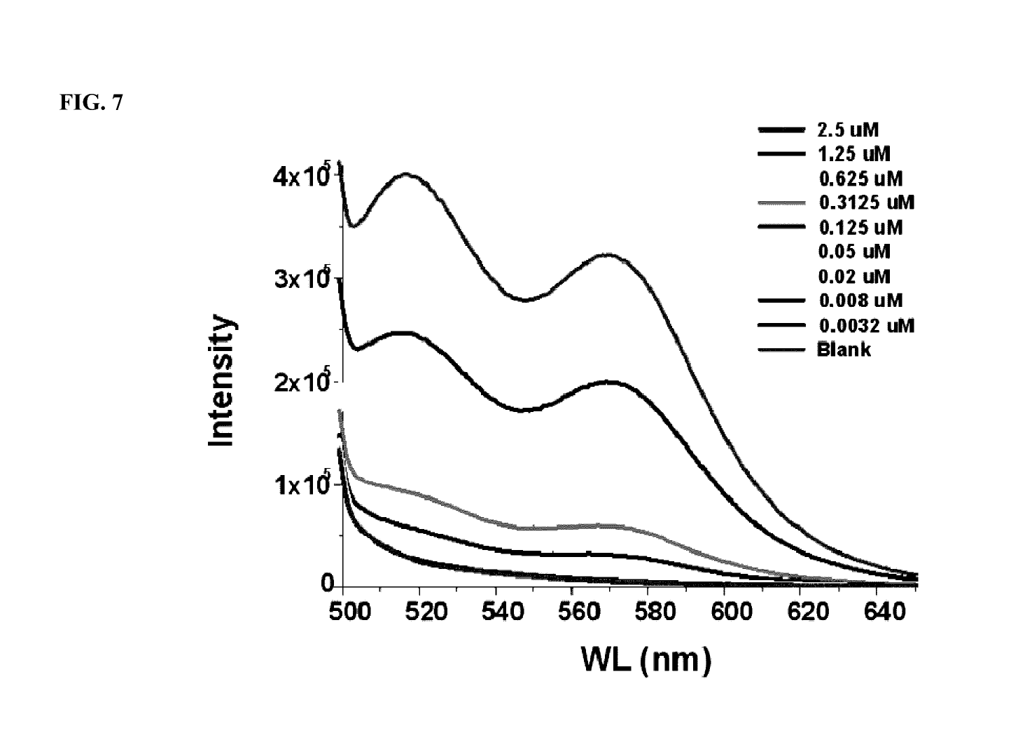

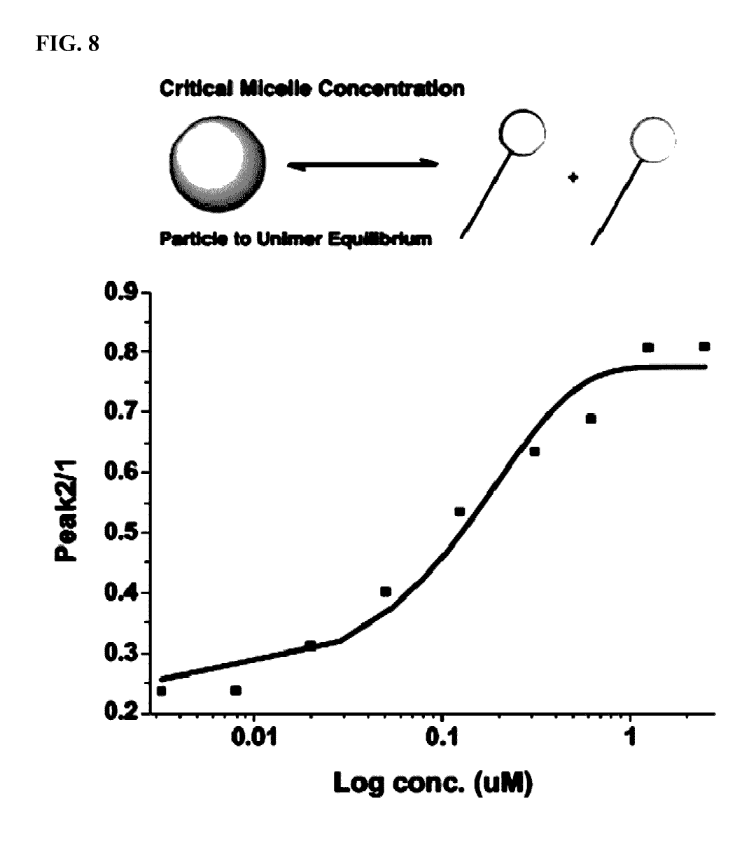

Dye-labeled self-assembled aggregates were formed from peptide-polymer amphiphiles. The micellar nanoparticles carried a single fluorophore or two fluorophores that created a FRET signal (FIGS. 6-7). A unique FRET signal was generated by establishing a particle to unimer equilibrium during synthesis (FIG. 8).

Enzyme-responsive fluorogenic peptide-programmed nanomaterials were designed for use as MRI contrast agents. FIG. 9 shows MRI-agent chelated that were directly incorporated into the polymer backbone of a peptide-programmed nanomaterial.

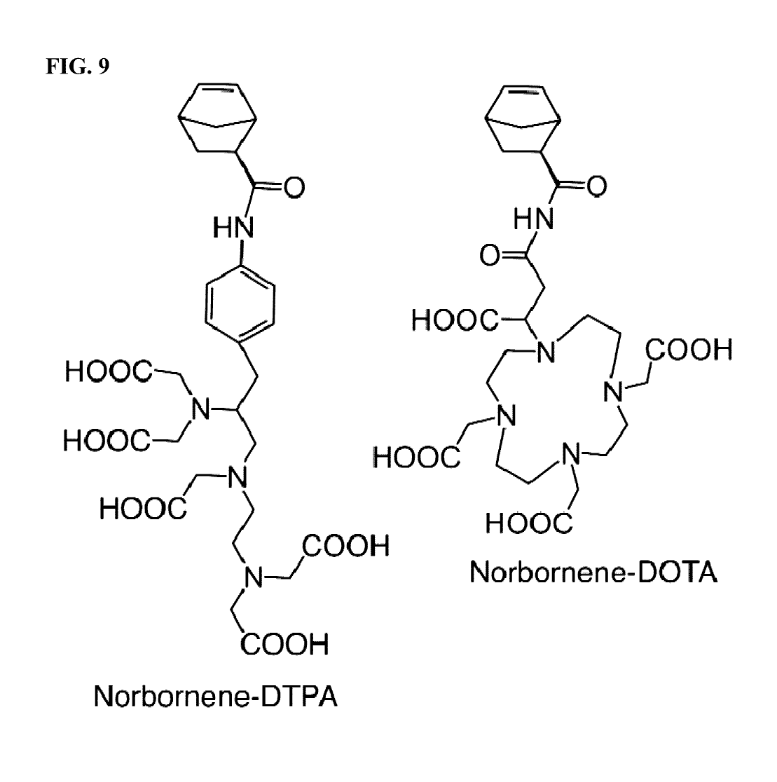

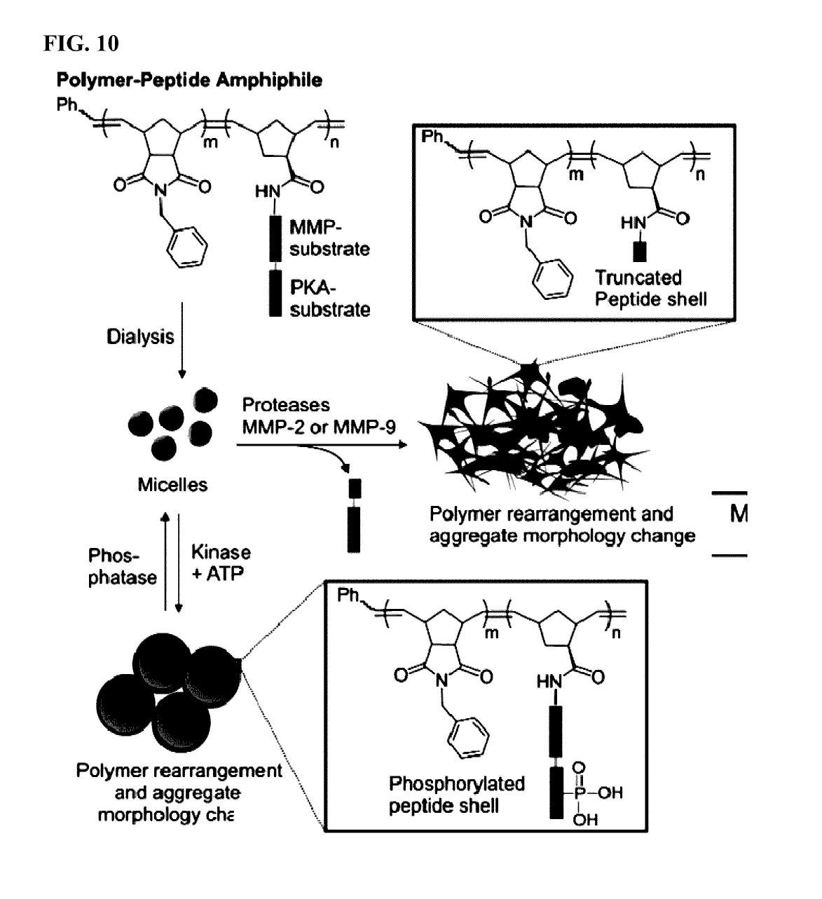

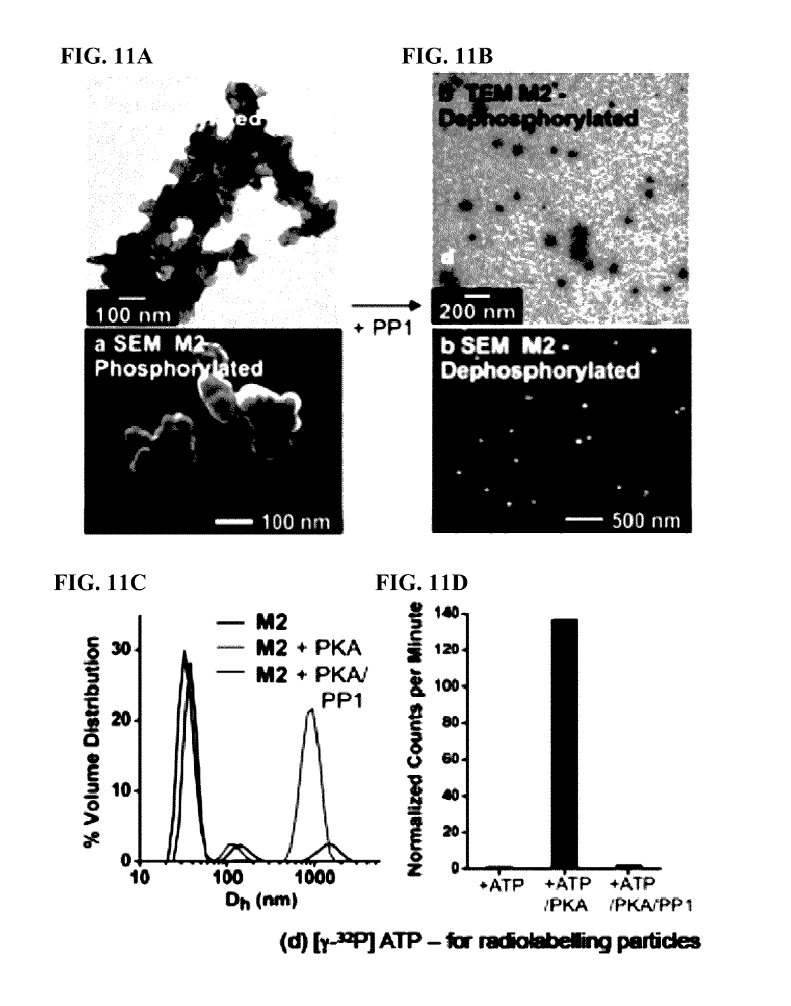

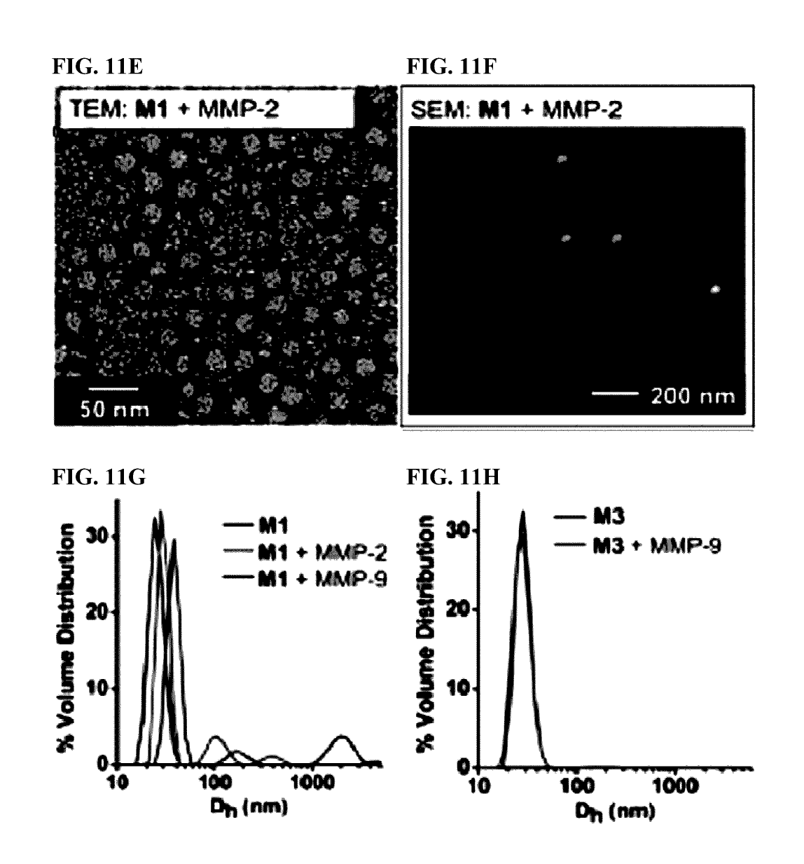

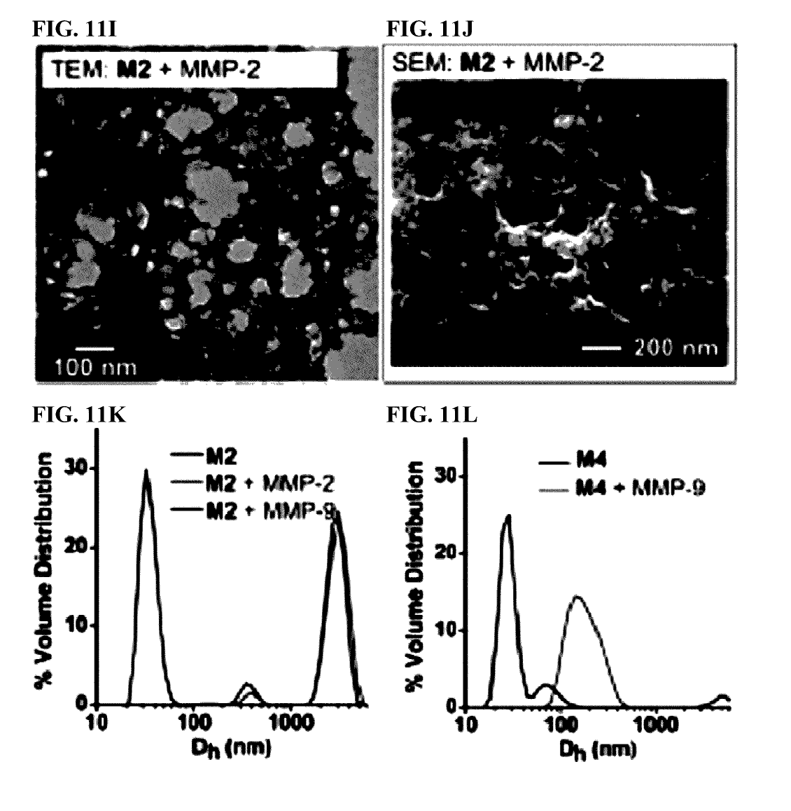

Multiple enzyme substrates added to a peptide-programmed nanomaterial. For instance, a peptide-polymer amphiphile was synthesized with a peptide that is recognized by proteases such as MMP2 and MMP9 and a peptide that is a substrate for phosphatases and kinases (FIG. 10). Protease cleavage of the micellar nanoparticle truncated the peptide shell to control polymer rearrangement and aggregate morphology change (FIGS. 11I-11L). ATP mediated kinase activity phosphorylated the peptide shell which induced a different type of polymer rearrangement and aggregate change (FIGS. 11A-11D).

### Example 2

### Peptide-Programmed Nanoparticles

The example illustrates enzyme-responsive fluorogenic peptide-programmed nanoparticles with detectable spectrophotometric properties unique to the particles and their aggregated state. These micelles are assembled from peptide-polymer amphiphiles (PPAs) labeled with either fluorescein or rhodamine. This is achieved by labelling otherwise similar block copolymer amphiphiles with each of the dyes. When mixed together, signals from the FRET-pairs can be utilized to detect particle assembly and hence enzymatic activity. Furthermore, we show FRET signals within the shell of the assembled micelles can be used to estimate particle stability (critical aggregation concentration) and enable a determination of intraparticle distances between amphiphiles in the micellar aggregates leading to elucidation of the packing arrangement of amphiphilic copolymers within the micelles.

### Introduction

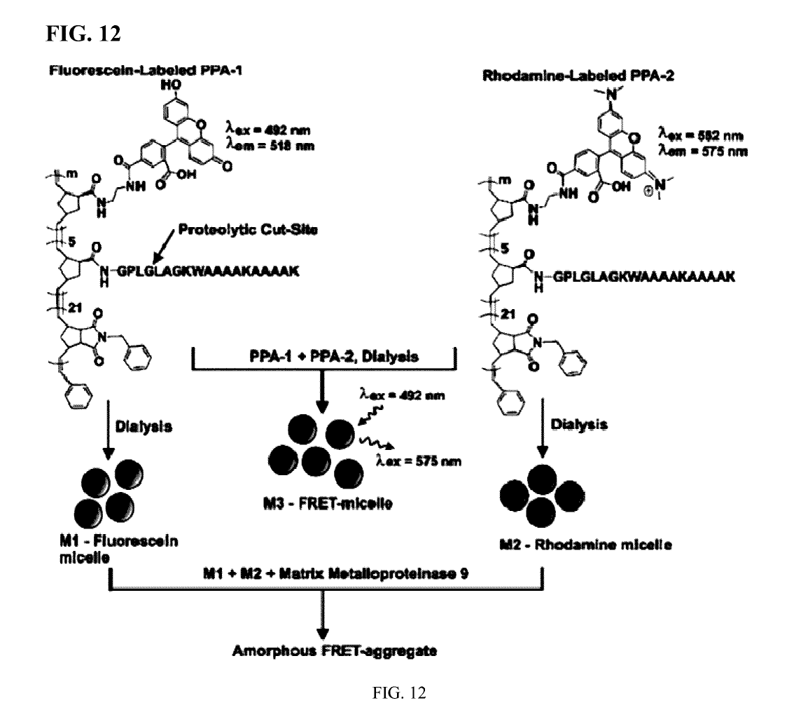

Enzymes are unique as biomarkers because they amplify detection events by catalytic turnover with selectivity that can be specific to given disease states (Saiki et al, Science, 1985, 230, 1350-1354; E. Engvall and P. Perlmann, Immunochemistry, 1971, 8, 871-874; L. Zhu and E. V. Anslyn, Angew. Chem. Int. Ed., 2006, 45, 1190-1196; Jiang et al., Proc. Natl. Acad. Sci., 2004, 101, 17867-17872). The specificity and diversity of reactions catalyzed by enzymes and their importance as signal-amplifying biomarkers make them exceptionally attractive as tools in the assembly and manipulation of nanoscale materials (M. E. Hahn and N. C. Gianneschi, Chem. Commun., 2011, 47, 11814-11821) In particular, nanoparticles capable of undergoing enzyme-programmed assembly, or morphology switches are of interest because unlike substrates such as fluorogenic oligopeptides, they can theoretically act as carriers of payloads that include specific molecular diagnostics and drugs. Although underutilized, enzymes have been harnessed as selective tools for the manipulation of nanoscale structures, a process that in itself constitutes a unique signaling event indicating enzyme activity (M. E. Hahn and N. C. Gianneschi, Chem. Commun., 2011, 47, 11814-11821; Von Maltzahn et al., J. Am. Chem. Soc., 2007, 129, 6064-6065; Ku et al., J. Am. Chem. Soc., 2011, 133, 8392-8395; Amir et al., J. Am. Chem. Soc., 2009, 131, 13949-13951; R. V. Ulijn, J. Mater. Chem., 2006, 16, 2217-2225). Such responses have proven detectable based on routine morphology analyses via methods including electron microscopy and light scattering. However, changes in nanoscale architecture will only be detectable in more challenging settings (e.g. in vivo) if the action of the enzyme results in an output signal unique to the assembly, such as a spectrophotometric response. To enable this, we have developed peptide-polymer amphiphiles (PPAs; Cui et al., Peptide Science, 2009, 94, 1-18; Chen et al., J. Am. Chem. Soc., 2008, 130, 13555-13557) linked to dyes (Behanna et al., J. Am. Chem. Soc., 2007, 129, 321-327) capable of undergoing efficient Förster Resonance Energy Transfer (FRET) for detecting structural properties and aggregation states of self-assembled enzyme-responsive nanoparticles. Herein, this concept is demonstrated for elucidation of particle stability, particle structure, and for monitoring enzyme-induced morphological transformations (FIG. 12).

### Results and Discussion

The PPAs utilized in these studies were designed as substrates for the cancer-associated enzyme, matrix-metalloproteinase 9 (MMP-9; Kessenbrock et al., Cell, 2010, 141, 52-67; Scherer et al., Cancer Metastasis Rev, 2008, 27, 679-690; D. G. Vartak and R. A. Gemeinhart, Journal of Drug Targeting, 2007, 15, 1-20). By utilizing this substrate as the polar head group of the copolymer, the micelle morphology and aggregation behaviour of the materials could be modified via peptide cleavage by MMP at the Gly-Leu peptide bond (FIG. 12). We reasoned that enzymatic reactions occurring within the shell of the particles would facilitate a dramatic reduction in the hydrophilicity of the peptide-block, and would subsequently result in changes to the overall architecture via the establishment of new equilibria for surfactant aggregation. The polymers were synthesized using ring-opening metathesis polymerization (T. M. Trnka and R. H. Grubbs, Acc. Chem. Res., 2001, 34, 18-29; Smith et al., Polymer Reviews, 2007, 47, 419-459) to generate block copolymers of a phenyl-modified norbornene as the hydrophobic block, a conjugatable NETS-ester for linkage through the amine terminus of the peptide, and a short block of primary amino-modified norbornene, for conjugation to dyes. As shown in FIG. 12, micelles of fluorescein labelled PPA-1 (M1) and rhodamine labelled PPA-2 (M2) were prepared by dialysis of solutions of the polymers in DMSO/DMF (1:1) against buffered water over 24 h. In addition, micelles were prepared from mixtures of the two PPAs to generate the FRET-micelle, M3.

The presently described methods allow an accurate determination of the arrangement of polymeric amphiphiles packed within micelles and to monitor structural changes induced by responses to enzymes. Moreover, this method is amenable to use in complex environments where other particulates may be present. Such solutions containing mixtures of particles are not easily amenable to analysis by light scattering or TEM image analysis. The distance dependence of FRET efficiency of appropriately paired dyes provides such a route and has been extensively utilized in biochemical systems (L. Stryer, Annu. Rev. Biochem., 1978, 47, 819-846; R. M. Clegg, Methods Enzymol, 1992, 211, 353-388; P. R. Selvin, Nat Struct Biol, 2000, 7, 730-734; J. R. Lakowicz, Principles of Fluorescence Spectroscopy, 1983). However, the use of FRET efficiency for elucidating structural parameters in supramolecular self-assembled systems has been surprisingly limited, despite its great potential in determining solution phase structures of multicomponent assemblies (J. R. Lakowicz, Principles of Fluorescence Spectroscopy, 1983). It has been used for studying interfacial regions in the assembly of nanoparticles and micelles (J. P. S. Farinha and J. M. G. Martinho, J. Phys. Chem. C, 2008, 112, 10591-1060; Schillen et al., J. Phys. Chem. B, 1999, 103, 9090-9103; Farinha et al., J. Phys. Chem. B, 1999, 103, 2487-2495). This example illustrates that in addition to enabling a direct measurement of exceptionally low CAC for micelles, and a geometrical determination of aggregation number (Schillen et al., J. Phys. Chem. B, 1999, 103, 9090-9103; Prazeres et al., Polymer, 51, 355-367). FRET-labeled PPAs can be utilized to sensitively monitor micellar nanoparticle response to enzymes. These parameters were determined by analysis of the distance dependence of FRET efficiency within polymeric micelles in which each amphiphile in the assembly is covalently end-labeled with one of two dyes in a pair. The result is micellar aggregates with dyes displayed on their surfaces.

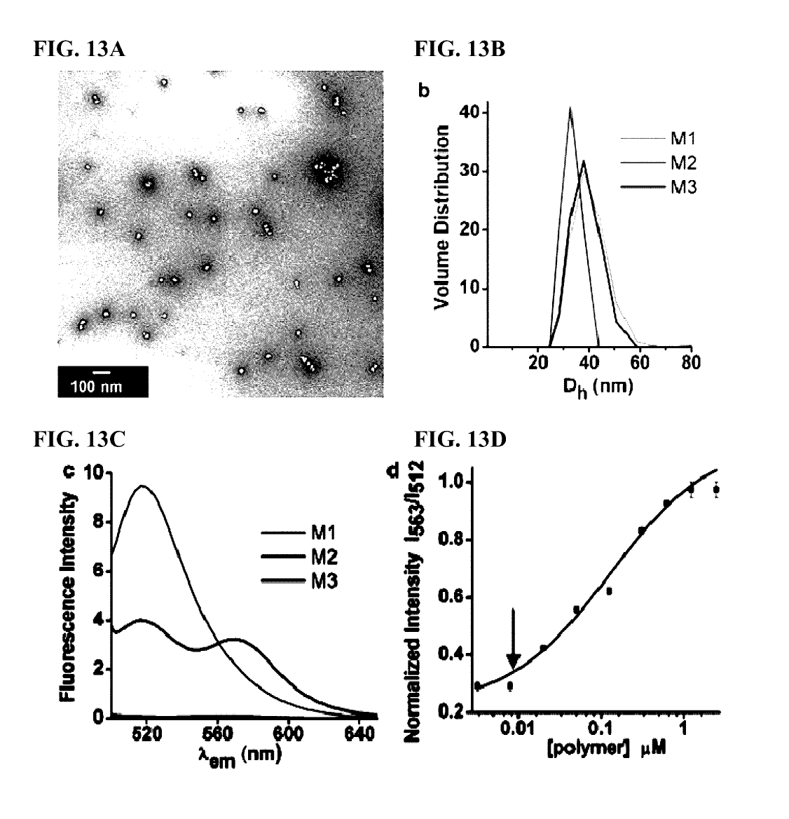

Initially, the fluorescence spectra and efficiency of FRET for a range of concentrations of PPA-1 and PPA-2 in the formation of micelles was studied (FIGS. 13A-13D). M1, M2 and M3, are 35-40 nm in diameter as characterized by TEM (FIG. 13A for M3) and by DLS (FIG. 13B). The two single dye labeled micelles have the expected spectroscopic properties, with a peak due to fluorescein at 512 nm for M1 and no observable fluorescence upon excitation at 470 nm for M2 (FIG. 13C). However, blending PPA-1 and PPA-2 to form M3 provides a mixed dye micelle with fluorescent properties indicative of a FRET pair within the Förster radius as evidenced by rhodamine fluorescence observable at 563 nm. At PPA concentrations above 1 μM we found that the ratio of the intensities of each peak maximum (I₅₆₃/I₅₁₂) is constant. This indicates the maximum FRET efficiency possible for this system (FIG. 13D). However, upon dilution of M3 over the range from 2.5 μM to 2 nM, a greater decrease in intensity of the peak at 563 nm (rhodamine) compared to 512 nm (fluorescein) is observed. Therefore, a CAC of 8 nM is assigned for M3 as the concentration at which the onset of a detectable FRET signal is observed. This is a generalizable approach providing an exceptionally sensitive and direct method for determining CAC. Such a labeling strategy for observing intact particles is especially useful in cases where they are particularly stable, limiting the utility of standard CAC determination assays using non-covalently associated solvochromatic dyes where limit of detection is significantly above CAC. Direct labeling of the polymers also means allows for observations in complex milieu without the need for additional dye additives.

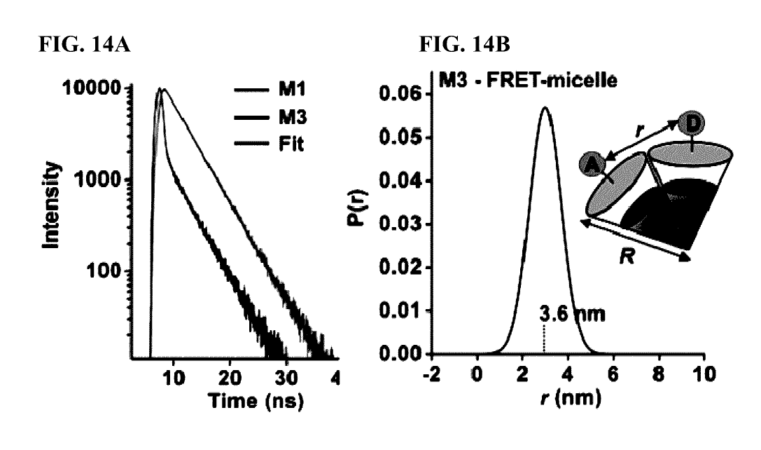

In addition to determining particle stability, we utilized the direct labeling strategy to elucidate structural features of the micelles. It has been shown that, if micelles are assumed to be assemblies of amphiphiles packed as cones with spherical bases, then the maximum integer number of amphiphiles in a micelle (Nₛₚₕ) can be directly calculated if the angle at the vertex of each cone (α) is known (Tsonchev et al., Nano Lett., 2003, 3, 623-626). We hypothesized that the angle α could be directly determined knowing the distance (r) between fluorescein and rhodamine, that are assumed to be located at the centers of each cone (or copolymer amphiphile) in a spherical micelle (FIGS. 14A-14B). The angle at the vertex (α) may be found via simple trigonometry because r is a chord between the centers of each spherical-based cone packed within micelles of radius, R. It follows that with experimentally measured values for R (TEM) and r (by FRET analysis), α can be calculated. Therefore, we assume that polymeric amphiphiles are close-packed cones that are not diffusing with respect to each other but are instead confined in restricted dimensions on the length scale relevant to FRET (J. P. S. Farinha and J. M. G. Martinho, J. Phys. Chem. C, 2008, 112, 10591-10601). Finally, each cone is end-labeled with donor or acceptor dyes that have fast rotational motion on the timescale of FRET.

The donor-acceptor (D-A) distance distribution was determined by analyzing the time-domain intensity decay of the donor (FIG. 14A; see, e.g., R. Lakowicz, Principles of Fluorescence Spectroscopy, 1983). Therefore, we fit the data as a summation of donor decays for all accessible D-A distances (FIG. 14B). For M3, we have R=15 nm, and r=3.6 nm +/−0.6, giving α=14°+/−2. Thus, we calculate Nₛₚₕ=241+/−60, taking account of the distribution about the mean distance between the donor and acceptor. This geometrically derived maximum aggregation number compares favorably with that determined from average particle molecular weight in solution by static light scattering (SLS) analysis of M3, which yields a weight average N_{agg}=209+/−0.82. Furthermore, SLS was used to determine N_{agg} of 159+/−0.34 for M1, and 304+/−2.3 for M2. It is clear that these values are on the same order, as expected for similar surfactants generating similar sized micellar nanoparticles. Indeed, the order of this aggregation number for this class of surfactant could be corroborated by counting particles in TEM images whereby the number of micelles per L was determined by mixing them with a stock solution of 20 nm gold nanoparticles. This FRET approach therefore provides a viable method for monitoring particle assembly and detailed structural features in PPA assemblies without requiring TEM or SLS.

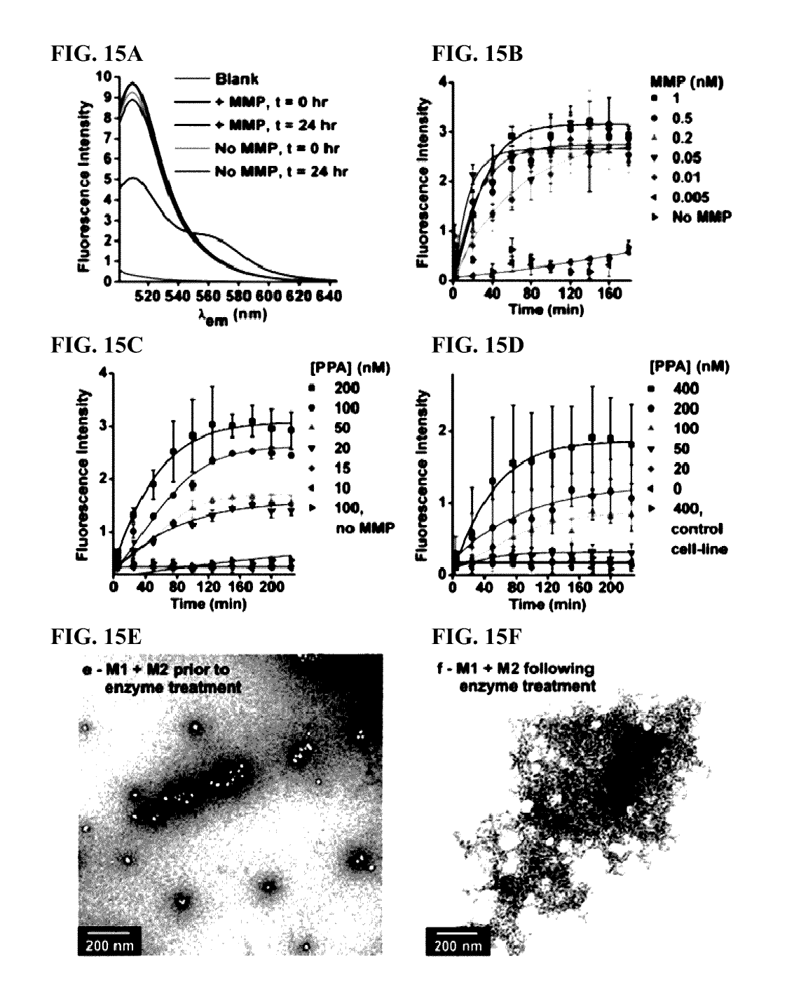

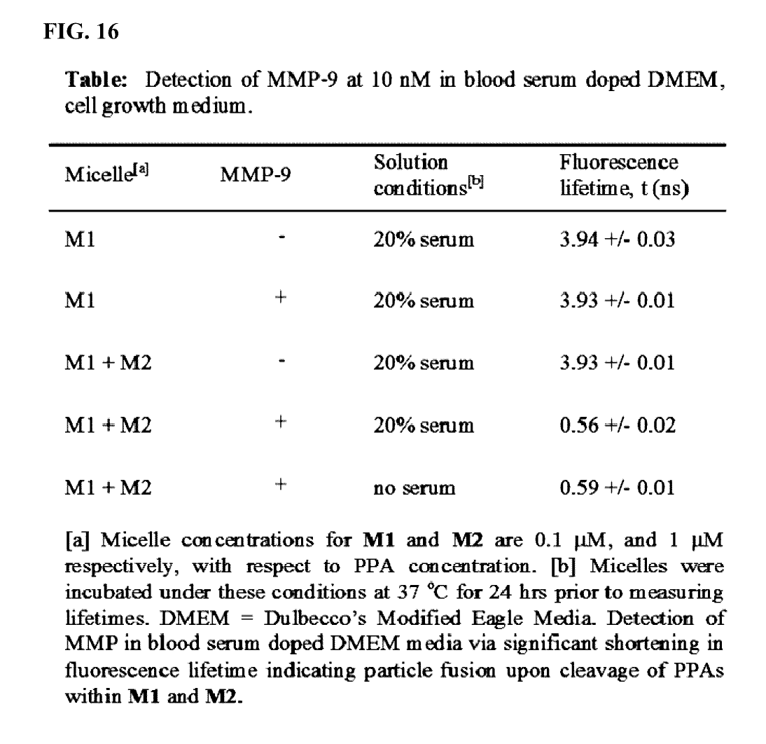

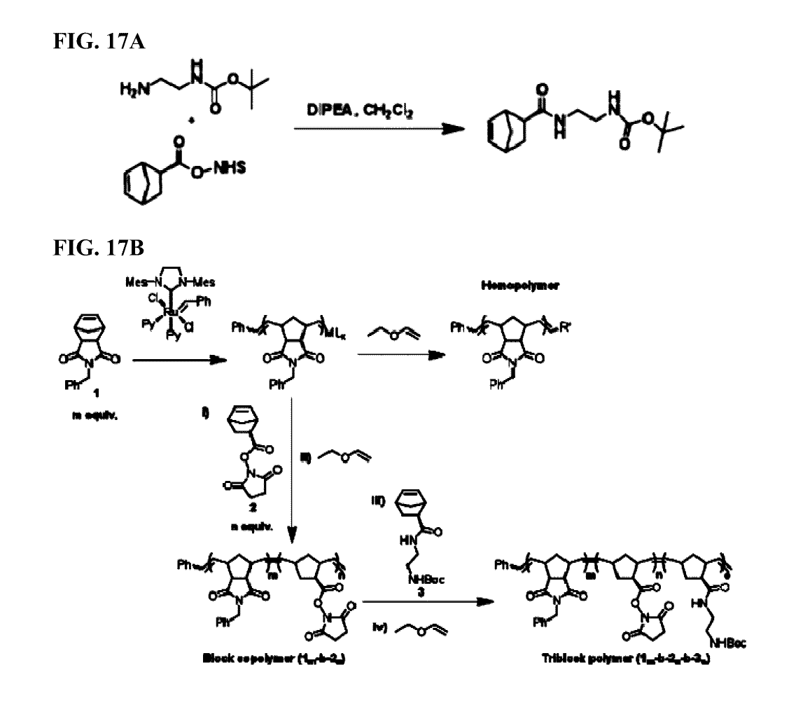

PPA-1 and PPA-2 were designed as substrates for MMPs. Therefore, we sought to study enzyme-induced rearrangement of the micelles, aiming to analyze the process via fluorescence spectroscopy in buffer solutions (FIGS. 15A-15F). Furthermore, to demonstrate the utility of FRET in analyzing micelle behavior in biological milieu, we examined their response to MMP-9 in blood serum doped samples of cell-growth media. Initial experiments were conducted to study enzyme kinetics on the micelle-based substrates. These were conducted by preparing a micelle from a PPA, end-labeled with fluorescein and conjugated to a peptide labeled with Dabcyl as a quencher, that when cleaved resulted in an ON-switch of fluorescence. These studies confirm that kinetics were similar on both particle-linked substrates and simple oligopeptide substrates. Next, we mixed M1 and M2 together with purified, commercial MMP-9 and observed the emission spectra over time (FIG. 15A) showing the formation of a new FRET-active species in solution as PPAs are cleaved and rearrange into aggregates containing both dyes. We note that peptide cleavage rates and formation of the new FRET signal, indicating aggregate formation, are similar and therefore consistent with them being concomitant processes. The peptide fragments could be quantified by HPLC (41% cleavage efficiency after 24 hrs), and characterized by MALDI. This low efficiency may be due to steric hinderance within the particle shells, or within the aggregates as they form during the reaction. Despite this, the particles are sufficiently susceptible to allow a complete shift to the aggregated species as evidenced by DLS. Furthermore, the FRET efficiency calculated from donor lifetimes is comparable to, but is reduced for these aggregates (Efficiency=85%) compared to M3 (Efficiency=93%). In addition, the relative in homegeneity of the aggregates compared to the control particles (M3) is observed by comparison of ratios of donor and acceptor intensities in FIG. 13C (1:0.8) vs. FIG. 15A (1:0.43). This is consistent with a less homogeneous distribution of donors and acceptors. Despite this slight decrease in FRET efficiency, the response is clearly observable down to 10 pM of MMP-9 for solutions containing 0.5 μM of micelles (FIG. 15B—concentration of micelles is with respect to PPA in each case). Furthermore, MMP-9 at 10 nM is observable down to 20 nM of PPA (FIG. 15C), a result that is consistent with the exceptional stability of these aggregates with CACs in the range of 10 nM. In addition, M1 and M2 underwent enzyme-induced aggregation when treated with a mixture of expressed MMPs (FIG. 15D). We note that these particles were designed to be responsive to both cancer-associated enzymes MMP-2 and MMP-9 as expected for the substrate sequence chosen. These experiments were performed by treating M1 and M2 with cell growth media containing MMPs excreted over 24 hrs from WPE1-NA45 cells, and present at 0.048 nM and 0.005 nM (MMP-2 and -9 respectively) as determined by a quantitative ELISA assay. In this case, substrate concentrations of 100 nM (with respect to PPAs) resulted in observable responses within 4 hr reaction time. Finally, this enzyme-induced rearrangement of PPAs was characterized by TEM, confirming the formation of a new aggregated species upon cleavage of the peptide sequence in M1 and M2 (FIGS. 15E-15F). It is this aggregated species that carries both dyes, in close enough proximity to allow spectroscopic characterization by FRET. To demonstrate the utility of the labeling approach in the detection of enzymes in more complex media, we mixed M1 and M2 in blood serum doped cell media samples and treated them with MMP-9 (FIG. 16). This resulted in an easily detectable and significant shortening of the fluorescence lifetime.

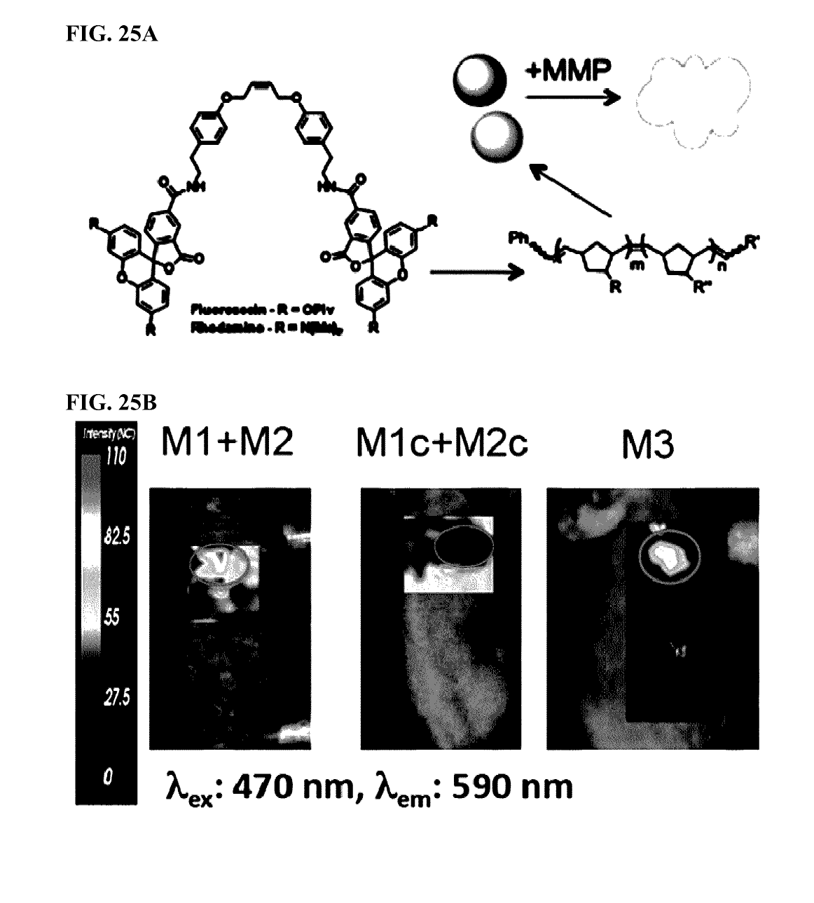

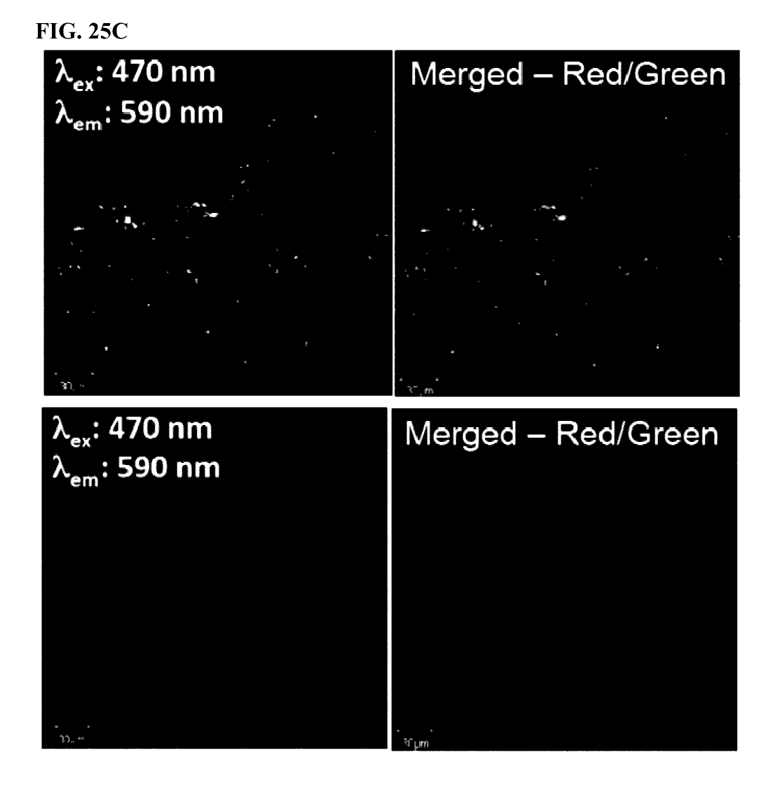

We injected PPAs into nude mice with HT1080 tumors which express MMPs to monitor the response of nanoparticles to disease-associated enzyme activity in vivo (FIG. 25A). We injection 4 nmoles to 20 μM of PPA-1 and PPA-2 in injectate (final concentration of PPAs was 1 nM) into the tail vein of the animal. We analyzed the mice 2 days post injection. Whole animal imaging showed the presence of M1+M2, M1c+M2c, and M3 nanoparticles in the tumors (FIG. 25B). Analysis of tumor sections showed fluorescence (FIG. 25C). The data shows in vivo assembly and collection of nanoparticles within tumor tissue expressing MMPs.

### CONCLUSIONS

This example illustrates the use of nanoparticles that undergo enzyme-induced changes in structure detectable in complex environments. This is a necessary step in the future implementation of enzyme-programmed materials in in vivo applications, in particular, where enzymatic signals are specific to given disease states including inflammation and metastasis (Scherer et al., Cancer Metastasis Rev, 2008, 27, 679-690). This is enabled by a labeling approach that provides critical information regarding particle structure and stability. Together, these studies are consistent with exceptionally stable micelles that show no detectable scrambling of PPAs when mixed together in the absence of MMP. Moreover, the enzymatic response constitutes a novel approach to the detection of enzymes whereby the stimulus induces detectable changes in nanoparticle morphology. This technology is for analyzing and utilizing the response of nanomaterials to enzymes as they undergo complex changes in structure (Samarajeewa et al., J. Am. Chem. Soc. 2012, 134, 1235-1242). Furthermore, by detecting changes in fluorescence lifetime induced by rearrangement of appropriately labeled polymers, these processes can be observed in complex milieu. This type of FRET-pair labeling strategy is useful to those wanting to monitor particle aggregation state in complex environments, where light scattering and electron microscopy suffer deleterious interference from other particulates and detritus. This labeling approach was also be used for monitoring the response of nanoparticles to disease-associated enzyme activity in vivo.

### General Methods

All reagents were bought from Sigma-Aldrich and used without further purification. Anhydrous toluene and dichloromethane were purified using a Dow-Grubbs two column purification system (Glasscontour System, Irvine, Calif.; Pangborn et al., Organometaliics, 1996, 15, 1518-1520). (N-Benzyl)-5-norbornene-exo-2,3-dicarboximide was prepared as described in a previous report (Ku et al., Journal of the ACS, 2011, 133, 8392-8395). 1-{[(2S)-bicyclo[2.2.1]hept-5-en-2-ylcarbonyl]oxy}-2,5-pyrrolidinedione was prepared as described by Pontrello et al. (Journal of the ACS, 2005, 127, 14536-14537.) (IMesH₂)(C₅H₅N)2(Cl)2Ru═CHPh was prepared as described by Sanford et al. (Organometallics, 2001, 20, 5314-5318). Polymerizations were performed under dry dinitrogen atmosphere with anhydrous solvents. MMP-9 was acquired from Calbiochem, as a solution in 200 mM NaCl, 50 mM Tris-HCl, 5 mM CaCl₂, 1 μM ZnCl₂, 0.05% BRIJ® 35 Detergent, 0.05% NaN₃, at pH 7.0. HPLC analyses of peptides were performed on a Jupiter 4u Proteo 90A Phenomenex column (150×4.60 mm) with a binary gradient using a Hitachi-Elite LaChrom L-2130 pump equipped with UV-Vis detector (Hitachi-Elite LaChrom L-2420). Gradient: (Solvent A: 0.1% TFA in water; Solvent B: 99.0% acetonitrile, 0.9% water, 0.1% TFA; gradient: 20% B from 0-4 minutes, 20-45% B from 4-34 minutes, and 45-75% B from 34-38 minutes, Flow rate: 1 mL/min). To confirm peptide molecular weight, MALDI-TOF mass spectrometry was performed on an ABI MALDI Voyager (equipped with ThermoLaser Science, VSL-337ND) using alpha-CHC matrix (alpha-cyano-4-hydroxycinnamic acid) (Agilent technologies). Polymer polydispersity and molecular weight were determined by size-exclusion chromatography (Phenomenex Phenogel 5u 10, 1K-75K, 300×7.80 mm in series with a Phenomex Phenogel 5u 10, 10K-1000K, 300×7.80 mm (0.05 M LiBr in DMF)) using a Hitachi-Elite LaChrom L-2130 pump equipped with a multi-angle light scattering detector (DAWN-HELIOS: Wyatt Technology) and a refractive index detector (Hitachi L-2490) normalized to a 30,000 MW polystyrene standard. Dₕ was determined by DLS on a Malvern Nano-ZS90. TEM images were acquired on carbon grids (Ted Pella, INC.) with 1% uranyl acetate stain on a FEI Tecnai G2 Sphera at 200 KV. Fluorescence measurements were taken on a SPECTRAMAX GEMINI EM (Molecular Devices). Fluorescence lifetime measurements were taken on a Horiba Fluorolog-3 fluorometer system. 1H (400 MHz) and 13C (100 MHz) NMR spectra were recorded on a Varian Mercury Plus spectrometer. Chemical shifts (¹H) are reported in δ (ppm) relative to the CDCl₃ residual proton peak (7.27 ppm). Chemical shifts (¹³C) are reported in δ (ppm) relative to the CDCl₃ carbon peak (77.00 ppm). Mass spectra were obtained at the UCSD Chemistry and Biochemistry Molecular Mass Spectrometry Facility.

### Peptide Synthesis

### Preparation of Peptide-1 for the Synthesis of PPA-1 and PPA-2, and Peptide-2 for Dabcyl Labeled Control

### General Solid Phase Synthesis Procedure

Peptides were synthesized by Fmoc-based solid phase peptide synthesis using preloaded Wang resins. Fmoc deprotection was performed with 20% piperidine in DMF (2×5 min) and coupling of the consecutive amino acid was carried out with HBTU and DIPEA (resin/amino acid/HBTU/DIPEA 1:3:3:4). The final peptide was cleaved from the resin by treatment with trifluoracetic acid (TFA)/Dichloromethane (DCM) (1:1) for 2 h. The resin was washed with DCM and ether and the combined organics were evaporated in vacuo to give an off white solid. Peptide 1 (SEQ ID NO: 4)sequence: Gly-Pro-Leu-Gly-Leu-Ala-Gly-Lys-Trp-Ala-Ala-Ala-Ala-Lys-Ala-Ala-Ala-Ala-Lys HPLC (retention time=28.8 min). MALDI-MS: Mass calculatedd: 1722.5; Mass observed: 1723.4. Peptide 2 sequence (SEQ ID NO:5): Gly-Pro-Leu-Gly-Leu-Ala-Gly-Lys(Dabcyl)-Trp-Ala-Ala-Ala-Ala-Lys-Ala-Ala-Ala-Ala-Lys HPLC (retention time=29.2 min). MALDI-MS: Mass calcd: 1973.5; Mass obs: 1974.7.

### Monomer Synthesis

tert-butyl-(2-((2S)-bicyclo[2.2.1]hept-5-ene-2-carboxamido)ethyl)carbamate

FIG. 17A shows monomer synthesis. To a stirred solution of 2 (538 mg, 2.28 mmol) and mono-Boc protected ethylenediamine (500 mg, 3.42 mmol) in dry CH₂Cl₂, was added DIPEA (794 uL, 4.56 mmol). The reaction was left to stir under a dinitrogen atmosphere for 48 hrs. The reaction mixture was washed twice with 10% HCl and the organic layer dried with MgSO₄, filtered and concentrated to dryness to give 562 mg 88% of 3 as a white solid. ¹H NMR (CDCl₃): δ (ppm) 1.28-1.34 (m, 2H, 1×CH₂, CH), 1.43 (s, 9H, CH₃), 1.67 (d, 1H, J=8 Hz, CH₂), 1.86-1.91 (m, 1H, CH), 1.99-2.02 (m, 1H, CH), 2.89-2.91 (m, 2H, 2×CH), 3.25-3.40 (m, 4H, 2×CH₂), 5.09 (bs, 1H, NH), 6.07-6.14 (m, 2H, 2×HC=CH), 6.40 (bs, 1H, NH). ¹³C NMR (CDCl₃): δ (ppm) 28.31, 30.40, 40.22, 40.75, 41.50, 44.57, 46.30, 47.05, 79.56, 135.94, 138.10, 156.96, 176.38. LRMS (ESI), 280.84 [M+H]⁺, HRMS, expected [M+Na]⁺: 303.1679. found: 303.1681.

### Polymer Synthesis

Backbone Copolymer (1₂₁-b-2₆-b-3₃)

FIG. 17B illustrates polymer synthesis. To a stirred solution of 1 (123 mg, 0.522 mmol) in dry CH₂Cl₂ (2 mL) cooled to −78° C. was added a solution of the catalyst ((IMesH₂)(C₅H₅N)₂(Cl)₂Ru═CHPh) (10 mg, 0.013 mmol) in dry CH₂Cl₂ (0.5 mL) also cooled to −78° C. After 5 min the cold bath was removed and the reaction was left to stir under nitrogen while warming to room temperature. After 40 min a 0.30 mL aliquot was removed and quenched with ethyl vinyl ether. After 25 min the polymer was precipitated by addition to cold MeOH to give the homopolymer as an off white solid. To the remaining reaction mixture a solution of 2 (35 mg, 0.148 mmol), in dry CH₂Cl₂ (1 mL) was added. The mixture was left to stir under N₂ for 40 min and a 0.30 mL aliquot was removed and quenched with ethyl vinyl ether. After 25 min the polymer was precipitated by addition to cold MeOH to give the block coopolymer as an off white solid. To the remaining reaction mixture, a solution of 3 (9.56 mg, 0.034 mmol), in dry CH₂Cl₂ (0.6 mL) was added. The mixture was left to stir under N₂ for 40 min followed by quenching with ethyl vinyl ether (0.100 ml). After 25 min the solution was concentrated to −1/3 the original volume then precipitated by addition to cold MeOH to give the copolymer as an off white solid. ¹H NMR of the polymer confirmed the absence of monomer (no olefin peak at 6.30 ppm) and the presence of broad trans and cis olefin peaks of the polymer backbone at 5.73 and 5.50 ppm, respectively.

SEC-MALS of polymers prior to peptide conjugation: Homopolymer of 1: Mn=5253, Mw/Mn=1.011, 1=21. Copolymer of 1-b-2: Mn=6725, Mw/Mn=1.050, 2=6. Triblock polymer of 1₂₁-b-2₆-b-3₃: Mn=7459, Mw/Mn=1.053, 3=3.

General method utilized in polymerization reactions. For analysis purposes a sample of the first and second blocks in the polymer was quenched prior to addition of the second and third monomer. This is used to confirm block size and is compared with weight fraction analysis of the copolymer by SEC-MALS.

### Representative Synthesis of Peptide-Polymer Amphiphile (PPA)

### Synthesis of PPA-1 and PPA-2

0.05 μmol of 1₂₁-b-2₆-b-3₃ was dissolved in 1 mL of Dimethylformamide (DMF), followed by addition of 1.2 equiv. of N,N-Diisopropylethylamine (DIPEA) and 1.2 equiv. of peptide. The reaction was stirred at room temperature for 22 hrs, followed by precipitation of the polymer by addition to cold methanol (1 mL). The precipitate was separated from the supernatant by centrifugation at 13,000 rpm. The precipitated peptide-triblock polymer product was then mixed with 12% TFA in 0.5 mL DMF for 2 hrs to remove the Boc protecting groups on the amine-functionalized block. The product was precipitated with cold ether (1 mL) followed by centrifugation at 13,000 rpm. The precipitated product was then dried and aliquoted into 0.5 mL DMF for generation of PPA-1 via addition of Fluorescein-NHS (0.54 mg, 1.1 μmol), and PPA-2 via addition of Rhodamine-NHS (1.8 mg, 3.4 μmol), each with 1.2 equiv. of DIPEA for 18 hr. The polymers were again precipitated by addition to cold ether (1 mL) followed by centrifugation at 13,000 rpm. dn/dc for the peptide-polymer conjugates is 0.179 as determined from peak analysis.

UV-Vis Determination of Dye Conjugated Efficiency to the Amine Block of 1₂₁-b-2₆-b-3₃

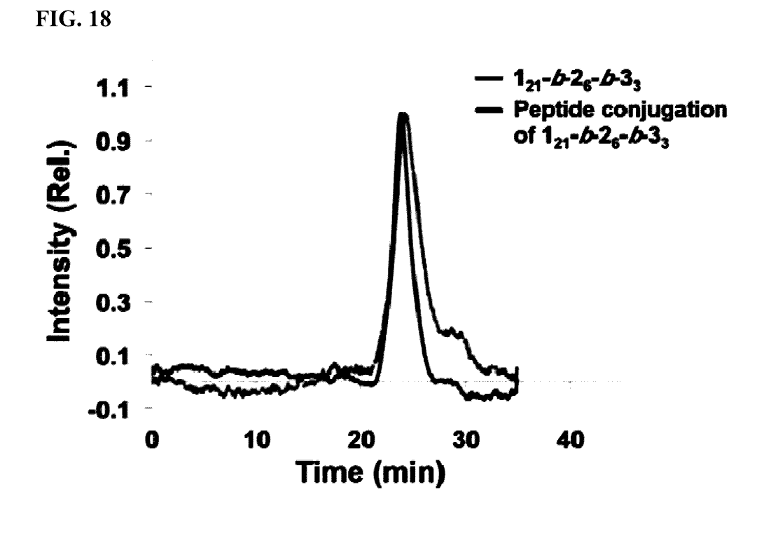

Dye conjugated efficiency was determined by calculating the concentration of peptides and dyes with extinction coefficient from UV-Vis measurement. The number of peptide conjugation was measured from SEC-MALS (see, FIG. 18) and the number of dye conjugation can be then calculated.

### Spherical Micelle Formation

Peptide-polymer amphiphiles (PPA-1 and -2; 0.25 mg, 16.3 μmol) were dissolved separately to generate M1 and M2, each in 70 μL of DMSO/DMF (1:1 ratio) followed by addition of 100 μL of sodium phosphate buffered water (40 mM, pH 8.0). This solution was then transferred to a 3,500 MWCO dialysis tubing and left for 3 days. The buffer was changed three times, once per day. For generation of M3 micelles, both PPA-1 and -2 (0.2 mg, 13 μmol) were mixed together and subjected to dialysis as described.

### Transmission Electron Microscopy (TEM)

Small (5 μl) aliquots of sample were utilized for TEM via standard procedure. Briefly, the sample was loaded onto grids (Ted Pella Inc.) that had previously been subjected to glow discharged using an Emitech K350 glow discharge unit and plasma-cleaned for 90 s in an E. A. Fischione 1020 unit. The sample grid was then transferred into a grid holder in a FEI Sphera microscope operating at 200 keV. Micrographs were recorded on a 2K×2K Gatan CCD camera.

### Enzyme Activation

To 5 μL of enzyme was added 0.4 μL of a 24 mM π-aminophenyl mercuric acetate solution in freshly prepared 0.1 M NaOH. The enzyme solution was heated at 37° C. for 2 hrs prior to use.

### Initial Study of Enzyme Kinetics on Micelluar Substrates

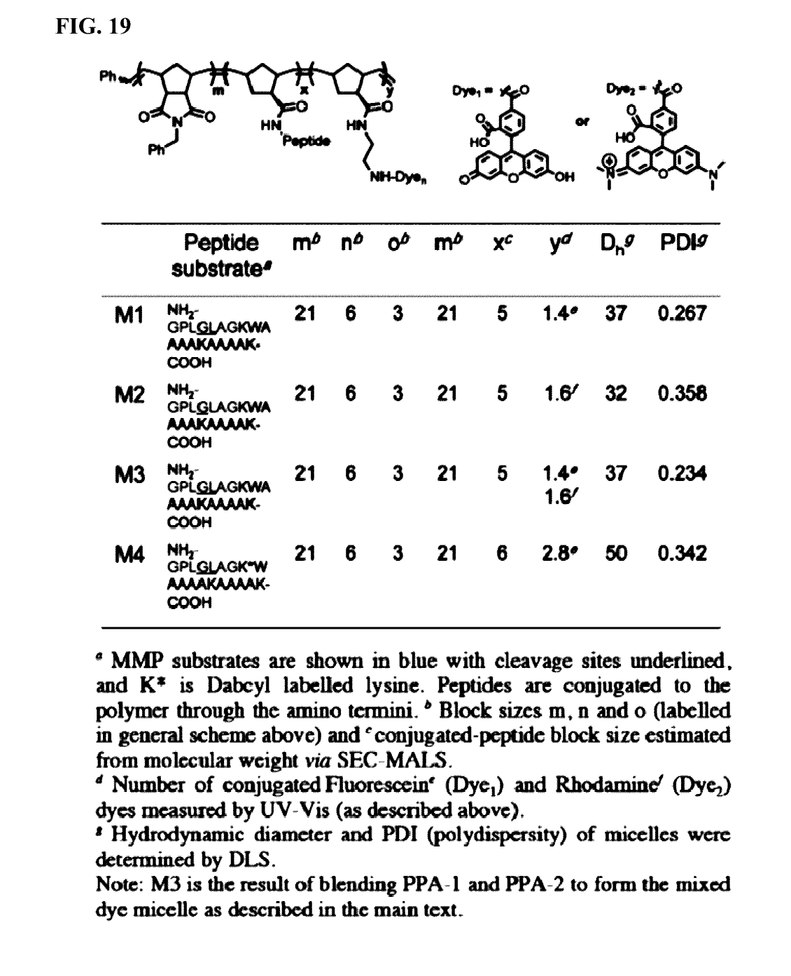

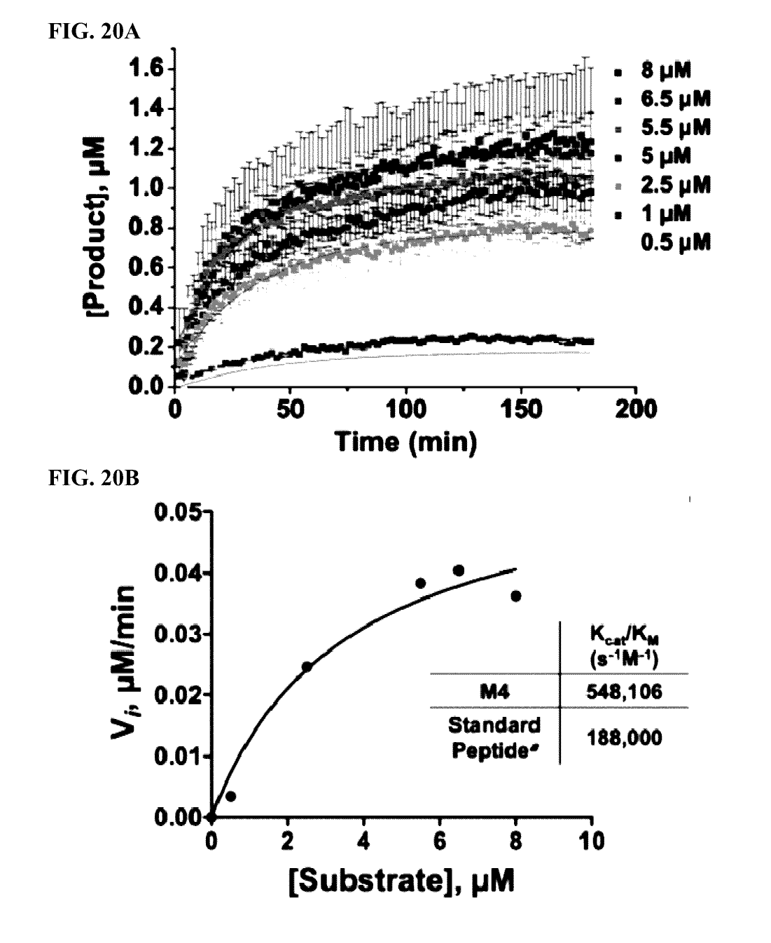

The kinetics of the MMP-9 driven cleavage of a micelle-based substrate was carried out using M4 (see, FIG. 19), which contained a peptide substrate with a Dabcyl label, and a polymer labeled with fluorescein. The fluorescein signal increase upon cleavage of the Dabcyl label was monitored. Different substrate concentrations (0.5, 1, 2.5, 5, 5.5, 6.5, and 8 μM, FIG. 20A) were reacted with MMP-9 (10 nM) from which initial rates of reaction were determined. The plot of initial rate (Vi) vs substrate concentration was fit to Michaelis-Menten kinetics (using PRISM) to obtain Kcat and KM showing this substrate for MMP-9 is comparable to standard substrate (FIG. 20B) (Kridel et al., Journal of Biol. Chem, 2001, 276, 20572-20578).

### MMP-9 Cleavage Efficiency Assay

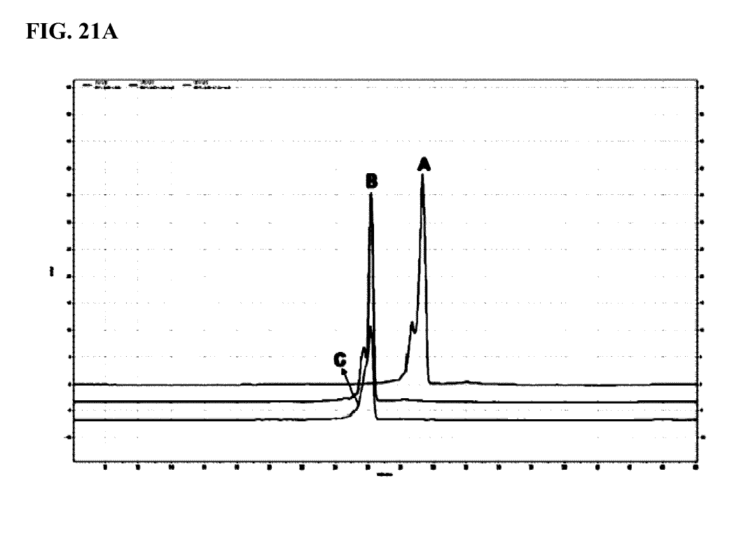

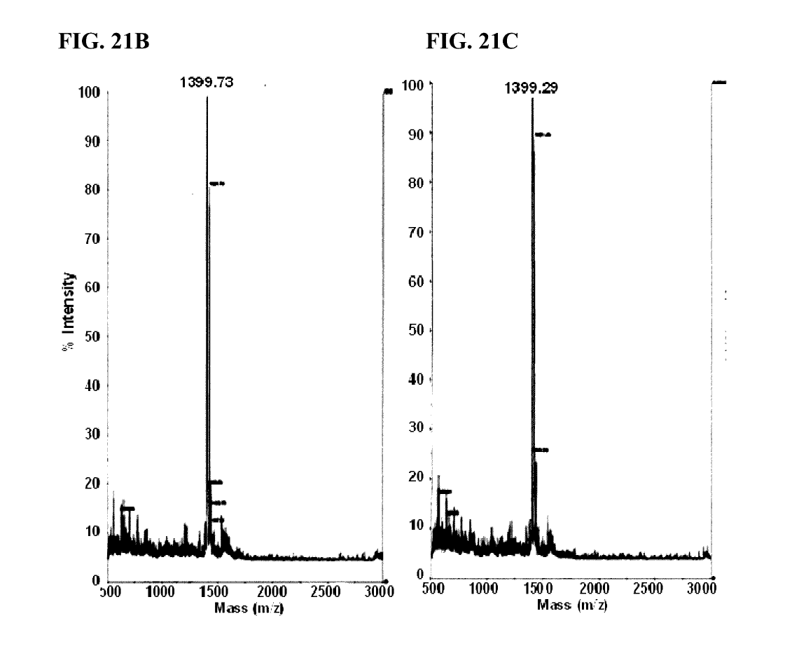

120 μM of M1 (with respect to peptide) and 120 μM of Peptide-1 were treated with MMP-9 (100 μU, 1.25 μL) for 24 hrs. The control was performed using 120 μM of Peptide-1 without treatment with MMP-9. These samples were then analyzed by RP-HPLC following inactivation of MMP-9 at 65° C. for 20 mins (FIG. 21A). Peak A is the intact, full length Peptide-1 without treatment with MMP-9, and Peak B is the large fragment of Peptide-1 cleaved by MMP-9 as confirmed by MALDI-MS shown in FIG. 21B, left. Peak C (FIG. 21A) is fragment resulting from the cleavage of M1 as confirmed by MALDI-MS (FIG. 21B, right. MMP-9 cleavage efficiency of Peptide-1 is quantitative according to the disappearance of the peak at 29 minutes. Therefore, MMP cleavage efficiency of M1 is estimated to be 41% (FIG. 21A) as a fraction of the area of Peak B. We note that it is possible this is a low estimate because particle aggregation may prevent all free peptide fragment from entering the RP column on HPLC. Alternatively, it is possible that steric hindrance reduces accessibility to the enzyme.

### Kinetics of FRET Fluorescence by Cell-Expressed MMP Enzymes

FIG. 15D shows the time course data for the development of the FRET signal from enzyme-driven M1/M2 aggregation utilizing cell-expressed MMP-2 and -9, from MMP overexpressing cell line, WPE1-NA45. MCF-7 was chosen as the control cell line. Both cells were seeded at concentrations of 1.6×10⁴ cells/well, in 96-well clear bottom culture plates. After 24 hrs, the cell medium was transferred to another 96-well black bottom plate for fluorescence measurements in the presence of M1 and M2 mixtures. M1 and M2 at various concentrations were used in this assay. WPE1-NA45 cells ecreted MMP enzymes were quantified by ELISA assays.

### Enzyme-Linked Immunosorbent Assay (ELISA) for Quantification of Enzyme

MMP-2 and MMP-9 ELISA kits were purchased from Invitrogen, Iinc. The procedure was carried out as per the manufacturer's standard instructions. Briefly, MMP standards from the kit and samples (cell medium from WPE1-NA45 and MCF-7 at time points of 0, 12, 24, 36 and 48 hrs) were added into the well strips and incubated for 2 hrs at room temperature. The solution was then discarded and washed four times. A solution of biotinylated MMPs was then added and reacted for an hour at room temperature. Following this, the solution was discarded and washed four times. To this was added a Streptavidin-Horse Radish Peroxidase solution for 30 min which was then discarded. The “Chromagen” solution was then added to the wells followed by another wash. The absorbance at 450 nm was measured after the addition of a “stopping” solution. Therefore, in this manner, cell-expressed MMP concentrations in the supernatant media as added to M1 and M2, were calculated with calibration by the MMP standards.

### Calculation of Nw^{agg} Via Particle Counting

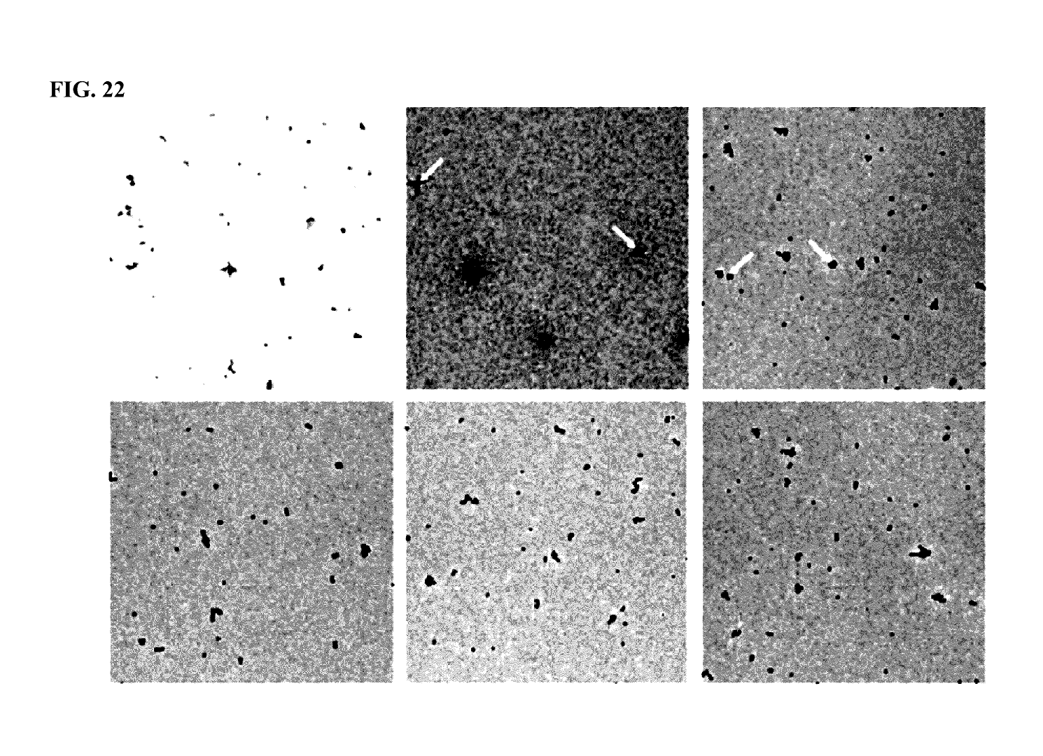

Micelle particle counting was performed via a 20 nm gold nanoparticle (Au NP) calibration utilizing TEM image analysis. 20 μL of M3 at a polymer concentration of 1.1 μM (as determined by UV-Vis), was mixed with 20 μL of 20 nm Au NPs at a concentration of 7×10¹⁴ particles/L. A total of 1243 M3 particles were counted, and 158 Au NPs were counted. FIG. 22 shows TEM images of particles from these solutions, with white arrows indicating the Au NPs. M3 is clearly discernable from the high contrast Au NPs as uranyl acetate stained organic material. Using this calibration method we determine M3 solutions contain 5.51×1015 particles/L. The aggregation number was determined to be 120, taking the particle count (5.51×10¹⁵ particles/L) and dividing by the polymer concentration in terms of molecules/L (6.624×10¹⁷). This is on the same order as determined by SLS and via the geometrical analysis described in the main text. Alternatively, we can use this method as a confirmation of polymer concentration in solution as determined by UV-Vis. In this approach, the particle/L count (5.51×10¹⁵) was multiplied by the weight average aggregation number (Nw^{agg}), 209 from SLS measurements, yielding 1.15×10¹⁸ surfactants/L. From this calculation we determine polymer concentration in solution to be 1.9 μM. Again, this is similar to that determined by UV-Vis (1.1 μM), which is the technique used to determine concentrations for CMC and enzymatic studies.

### Micellar Molecular Weight Determination Via Static Light Scattering (SLS)

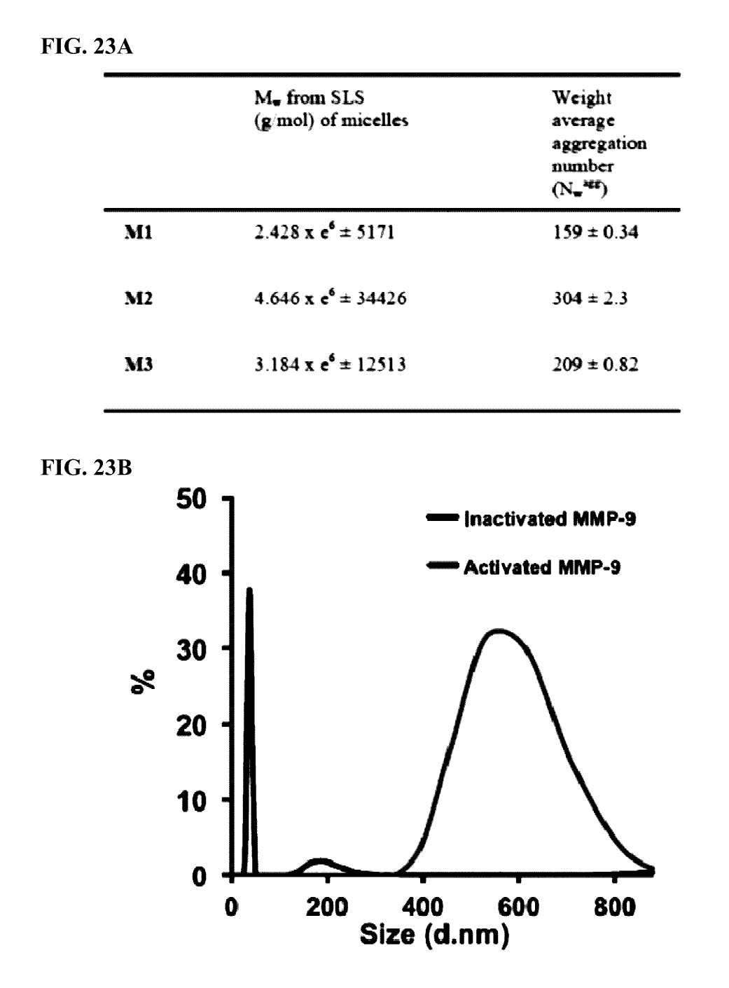

M1, M2 and M3 micellar average molecular weight were measured on a Wyatt Dawn Heleos-II multi-angle light scattering instrument in batch mode. M1, M2 and M3 micellar molecular weights were measured as 2.428×10⁶ g/mol, 4.646×10⁶ g/mol, and 3.184×10⁶ g/mol, respectively. These micellar molecular weights were then divided by polymer molecular weight (15,270 g/mol, FIG. 16) and micellar aggregation numbers were obtained as 159, 304 and 209 for M1, M2 and M3 micelles (FIGS. 23A-23B). This is further confirmation that aggregation numbers are on the order of 200 per particle for this type of surfactant, generating micelles on the scale of approximately 30-40 nm in diameter.

### Maximum Aggregation Number Calculated Knowing the Angle at the Vertex of Each Spherical Based Cone

The following equation was used to calculate N^{sph} and is derived from Tsonchev et al., Nano Lett., 2003, 3, 623-626:

$${N}_{sph} = Int ( \frac{2 π}{3 ( arccos ( \frac{cos α}{2 {cos}^{2} \frac{α}{2}} ) ) \cdot π} )$$

### Distance Distribution via Fluorescence Lifetime Measurement

Here, we have considered a range of D-A distances where the distance is expressed as a probability function P(r) distributed along the r axis (J. R. Lakowic, Principles of Fluorescence Spectroscopy, Springer, New York, 2006). A Gaussian distribution was used to describe the distance distribution, as in the equation below:

$$P ( r ) = \frac{1}{σ \sqrt{2 π}} exp [ - \frac{1}{2} {( \frac{\overline{r} - r}{σ} )}^{2} ]$$

In this equation r̅ is the mean of the Gaussian with a standard deviation of σ. The distance distribution is described by two standard deviations from the mean, with the probability of finding donor and acceptor within this range is 95.4%. The donor intensity decay is a summation of the intensity decays for all accessible distances, and is written as:

$$\begin{matrix}{I}_{DA} ( t ) = {\int }_{r = 0}^{∞} P ( r ) {I}_{DA} ( r , t ) d r \\ = {I}_{D}^{0} {\int }_{r = 0}^{∞} P ( r ) exp [ - \frac{t}{{τ}_{D}} - \frac{t}{{τ}_{D}} {( \frac{{R}_{0}}{r} )}^{6} ] d r\end{matrix}$$

This expression indicates that the intensity decay for an ensemble of flexible D-A pairs is given by the weighted average of the decays for each D-A distance. From this analysis, the distance distribution is calculated as 3.6±0.61 nm shown in FIGS. 14A-14B and written as r.

The lifetime in M3 (τ_{DA}) was then calculated from the standard treatment of FRET efficiency (E):

$$E = \frac{{R}_{0}^{6}}{{R}_{0}^{6} + {r}^{6}}$$

where R₀ is the Förster distance for the fluorescein and rhodamine pair, applied as 55 Å in this work given the assumption that rotation of the dyes is free and that therefore the orientation factor, κ²=2/3 (J. R. Lakowic, Principles of Fluorescence Spectroscopy, Springer, New York, 2006; Hochstrasser et al., Journal of Biol. Chem., 1989, 264, 19495-19499). The transfer efficiency can then be used to calculate the lifetime of the donor-acceptor (τ_{DA}):

$$E = 1 - \frac{{τ}_{DA}}{{τ}_{D}}$$

In this work, lifetimes of Fluorescein-Rhodamine labeled micelle (M3) and Fluorescein-labelled micelle (M1) were obtained as 0.29 ns and 3.98 ns respectively from fluorescence lifetime measurements (see, FIGS. 14A-14B).

DLS of M1 and M2 with and without Activated MMP-9

M1 and M2 micelles at 500 nM each with respect to PPA, were mixed with activated MMP-9 (see above for enzyme activation details) and non-activated MMP-9 (10 nM) at 37° C. for 24 hrs. DLS measurements were then taken (FIGS. 23A-23B).

Relative FRET efficiency for M3 may be expressed as the ratio of the intensity of the Rhodamine emission peak compared to the Fluorescein emission peak giving 0.8:1 (Rhodamine:Fluorescein). By contrast, FRET efficiency of the aggregated species produced when M1/M2 micelles were mixed with MMP-9 enzymes (FIG. 15A) is 0.43:1 (Rhodamine:Fluorescein). This can be contrasted against the efficiency of FRET from the lifetime (see equation above) of the donor for M3 found to be 92%. This compares to the aggregates with efficiencies of 85%. These data are consistent with a higher background of donor fluorescence in the absence of acceptor.

### M3+ MMP9

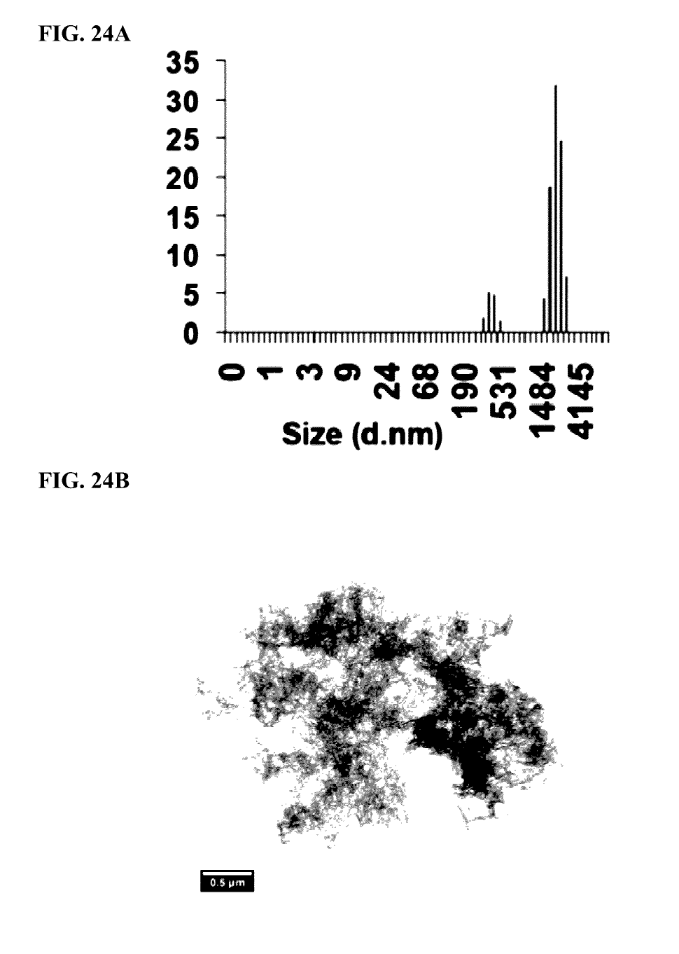

This experiment confirmed that M3 would undergo the same transformations in response to MMP-9 as M1 and M2. Micelles at 0.5 μM with respect to PPAs were mixed with MMP-9 (10 nM) for 24 hrs. FIGS. 24A-24B show DLS and TEM data for the formation of micron scale aggregates as for M1 and M2.

### Example 3

### DNA-Programmed Nanoparticles

This example illustrates a DNA-programmed micelle design and a method of synthesis. This example also illustrates a method of programming amphiphilicity to reversibly access various morphologies.

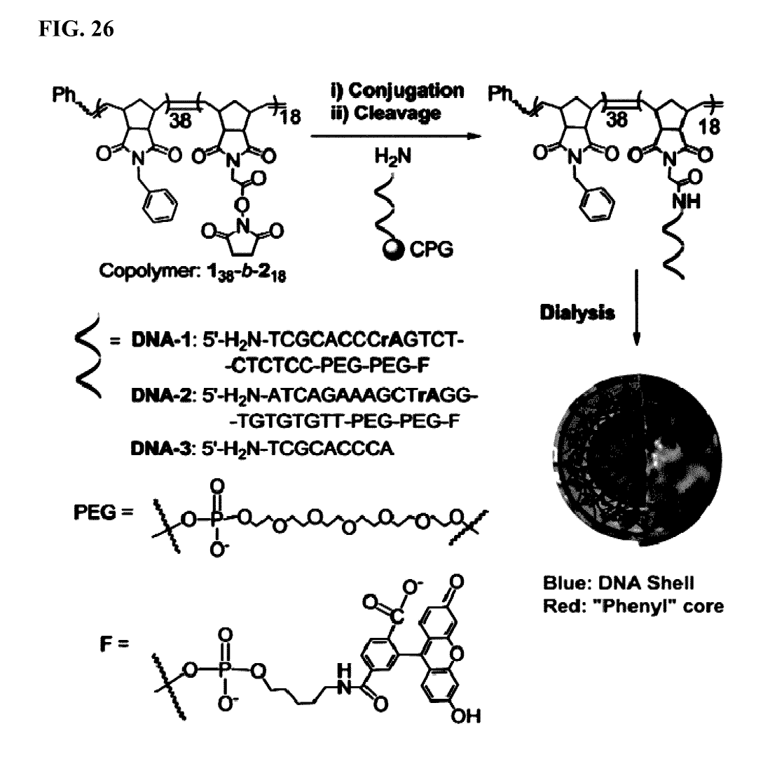

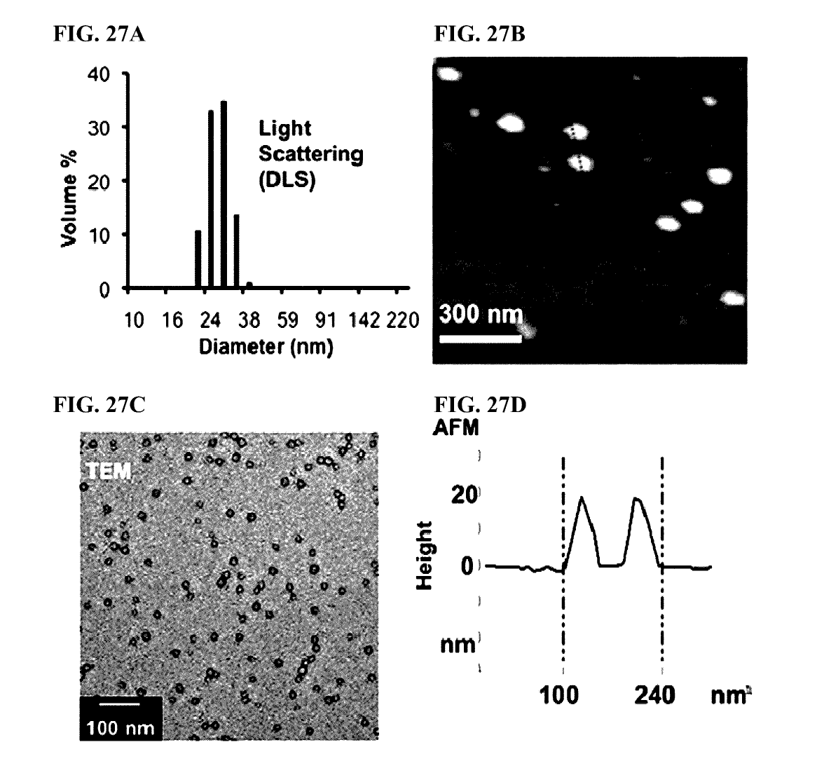

DNA-programmed nanomaterials were created from DNA-polymer amphiphiles (DPAs). Details are described in references such as Alemdaroglu F E and Herrmann A, Org. Biomol. Chem., 2007, 5, 1311-1320; Li et al., Nano. Lett., 2004, 4, 1055-1058; Chien et al., Angew. Chem., 2010, 49, 5076-5080; Thompson et al., Nano. Lett., 2010; Chien et al., Chem. Comm., 2010; and Chien et al., Small, 2011. The DNA sequence of a DPA is recognized by a sequence-specific enzyme that cleaves the DNA strand. FIG. 26 shows embodiments of a DNA-programmed micelle design. A copolymer 1₃₈-b-2₁₈ is conjugated with a DNA fragment, such as DNA-1, DNA-2 and DNA-3 (FIG. 26). The DNA-polymer amphiphile can include other moieties such as PEG or F. The DNA-polymer undergoes cleavage by DNA enzymes such nucleases, exonuclease, nicking endonucleases and DNAymes to form a micelle. The micelle comprises a DNA shell and a phenyl core. FIGS. 27A-27D show analysis of the DNA-programmed micelles.

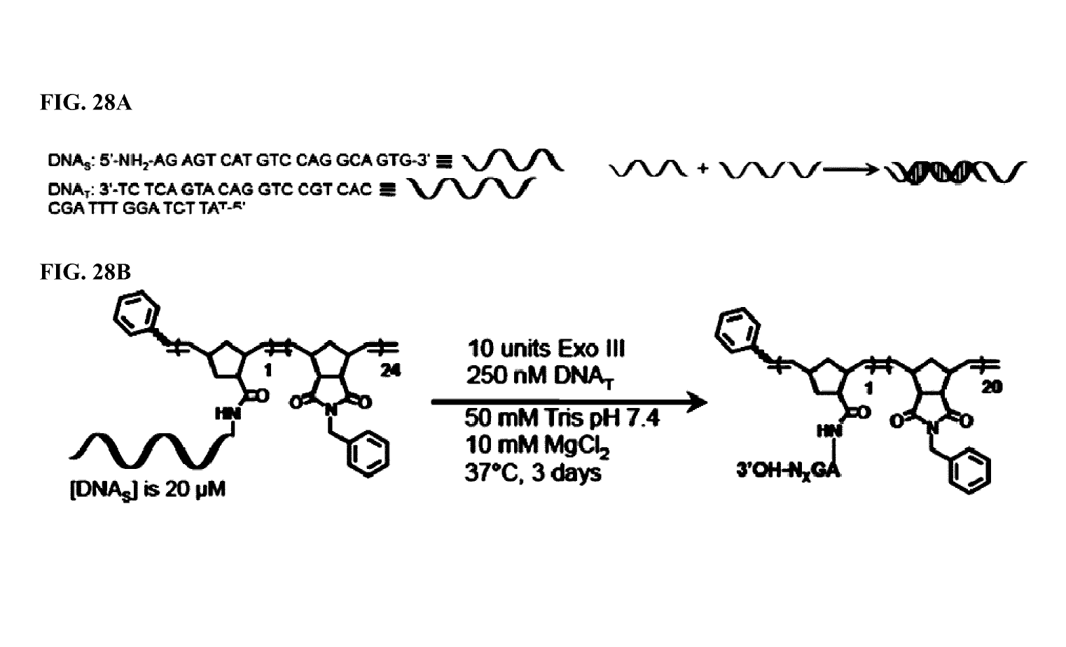

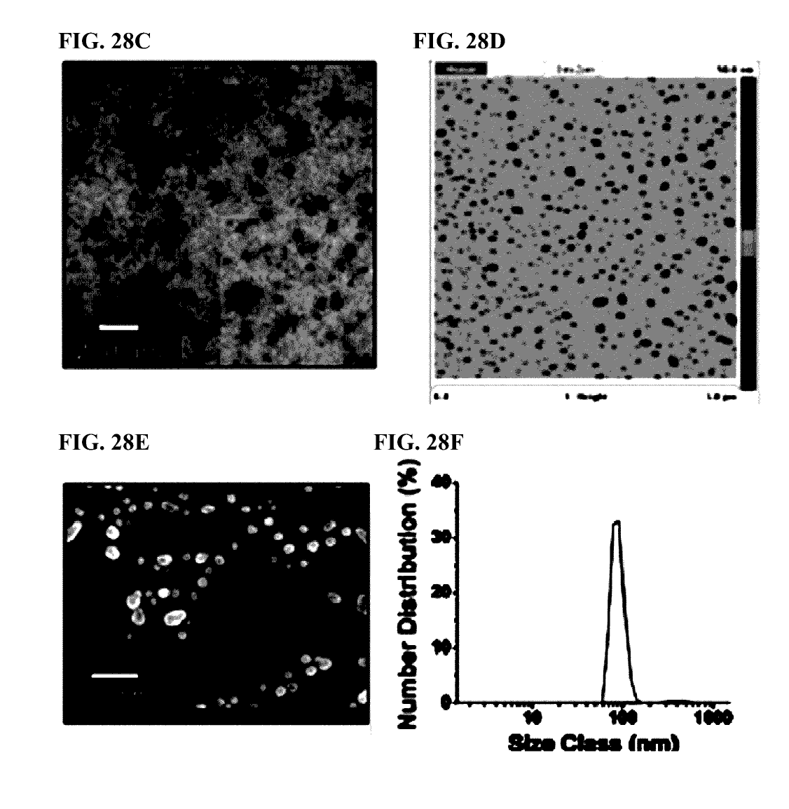

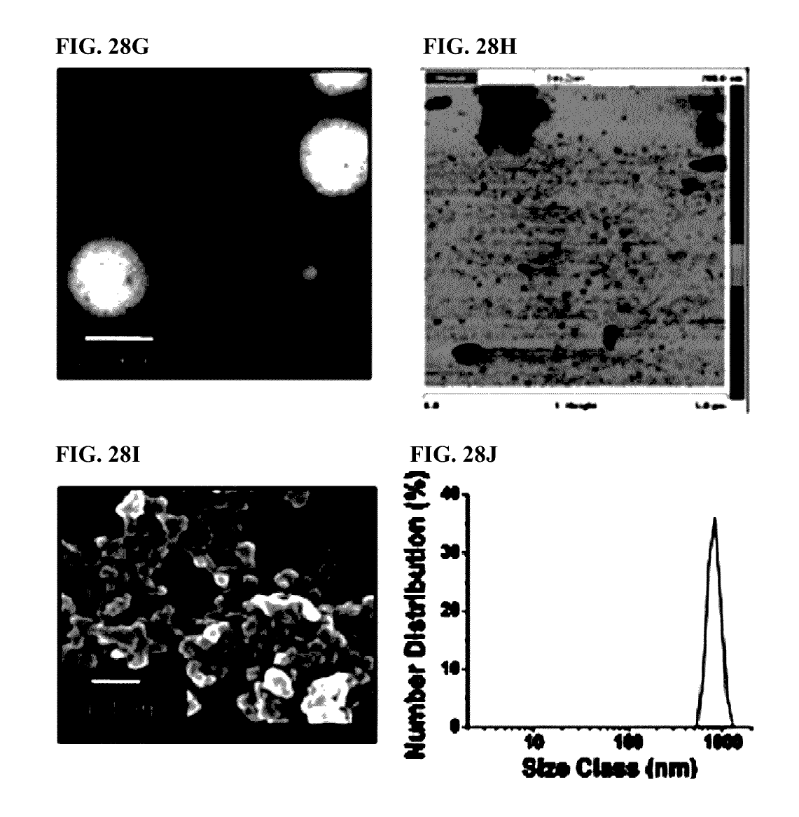

FIGS. 28A-28J show another example of DNA-programmed nanomaterial. DNAₛ is conjugated to the polymer to form the DPA. DNAₜ is complementary to DNAs and anneals to it to form double-stranded DNA. Exonuclease III recognizes the DNA sequence and cleaves it. The cleavage facilitates the formation of nanoparticle aggregates. FIG. 28A shows the DNA sequences. FIG. 28B shows the reaction used to form micelles and aggregates with DNAₛ based nanomaterial. FIGS. 28C-28J show images of the micelles (FIGS. 28C-28F) and aggregates (FIGS. 28G-28J).

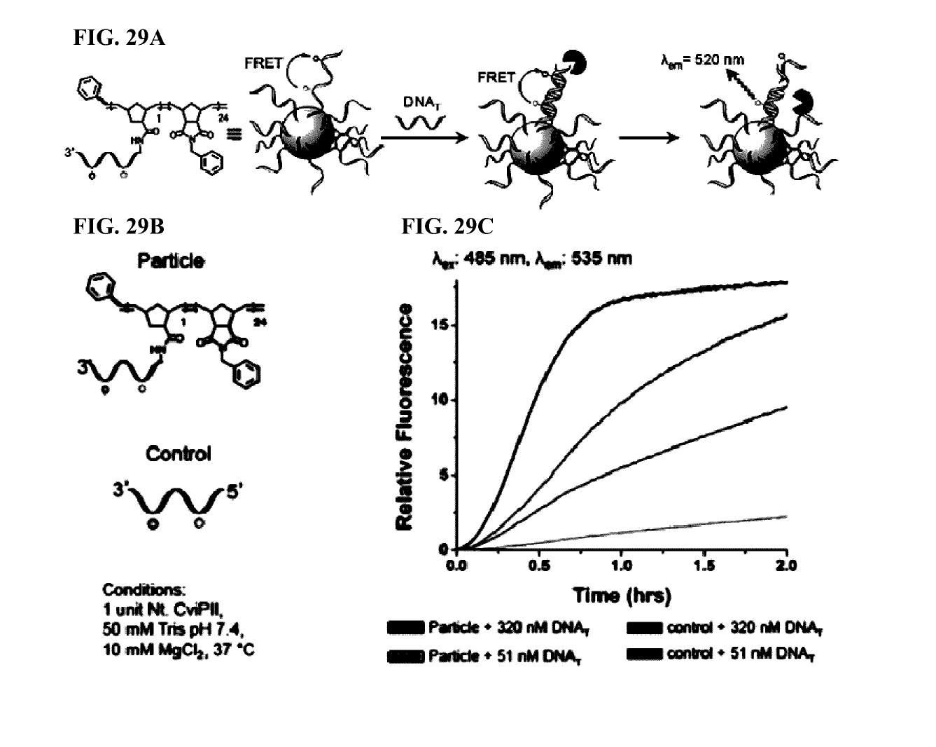

FIGS. 29A-29C show another example of DNA-programmed nanomaterial. DNAₛ of the DPA is labeled with a donor fluorophore (fluorescein) and an acceptor fluorophore (rhodamine) that are positioned to undergo FRET. The resulting micelle is incubated with DNAₜ that is complementary to DNAₛ. A nicking enzyme (e.g., Nt. CviPII) that specifically cleaves the annealed DNA strand releases the acceptor fluorophore, which allows the donor fluorophore to emit fluorescence.

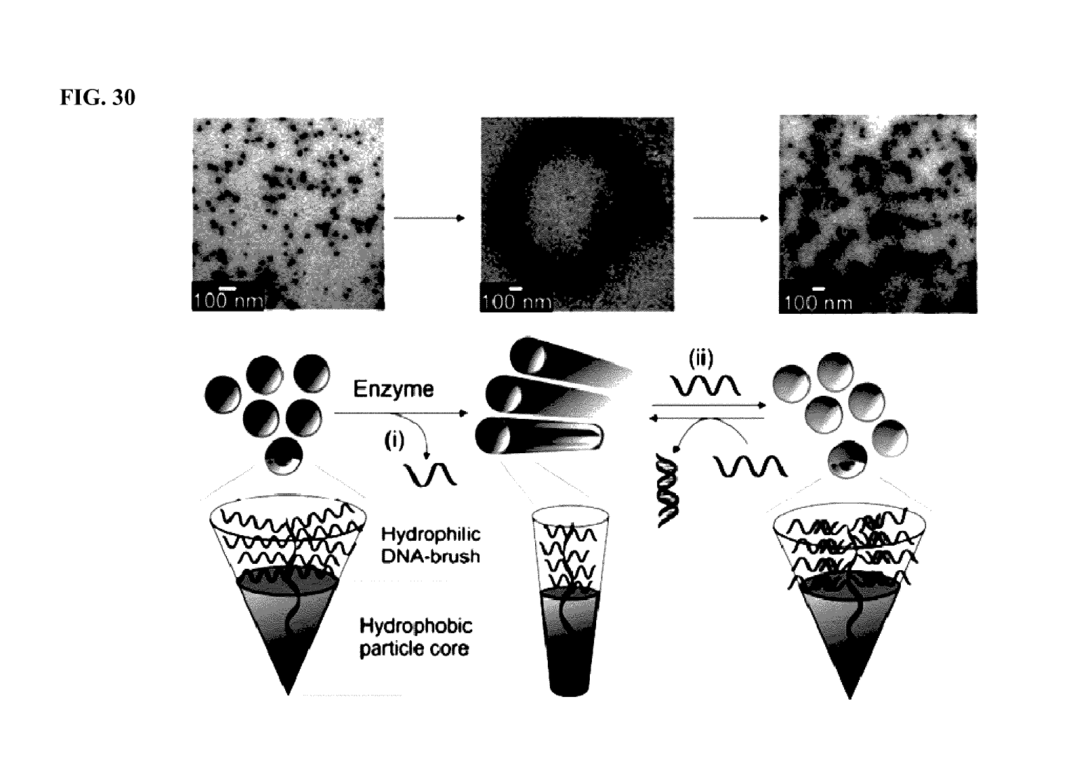

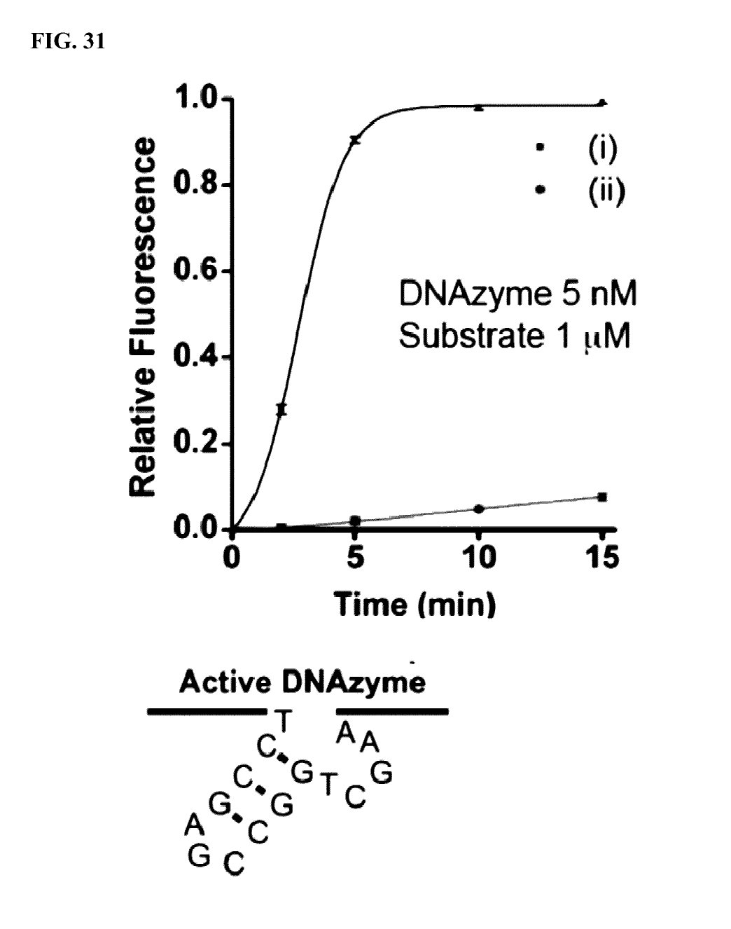

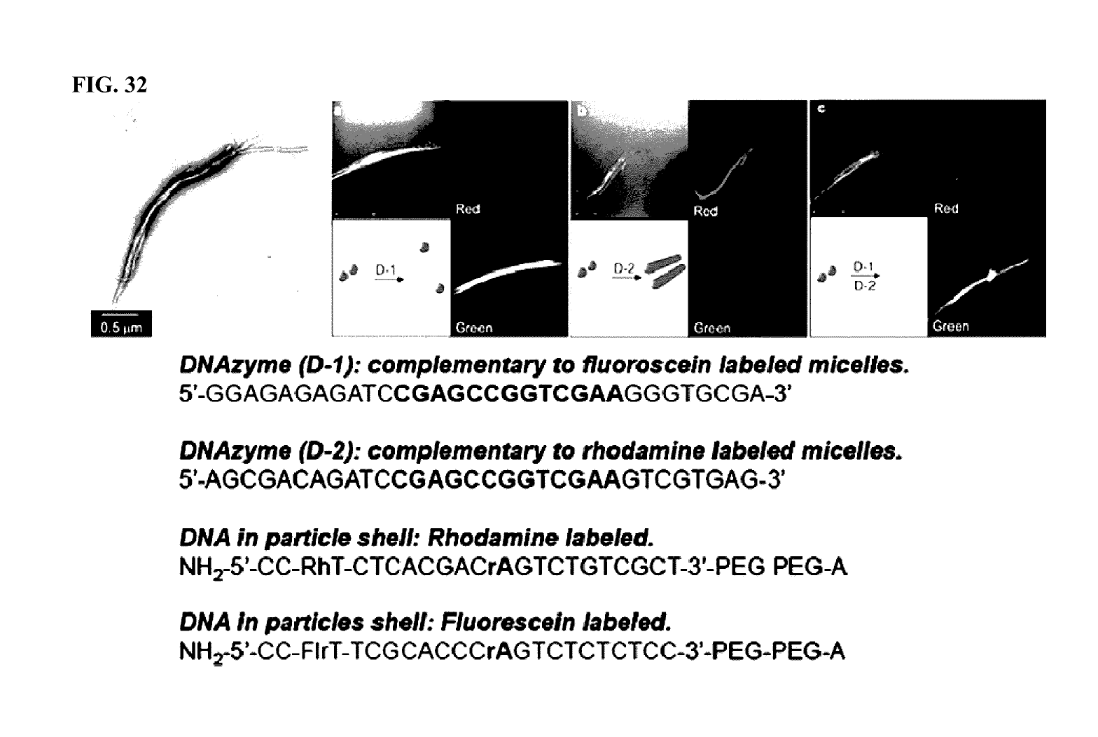

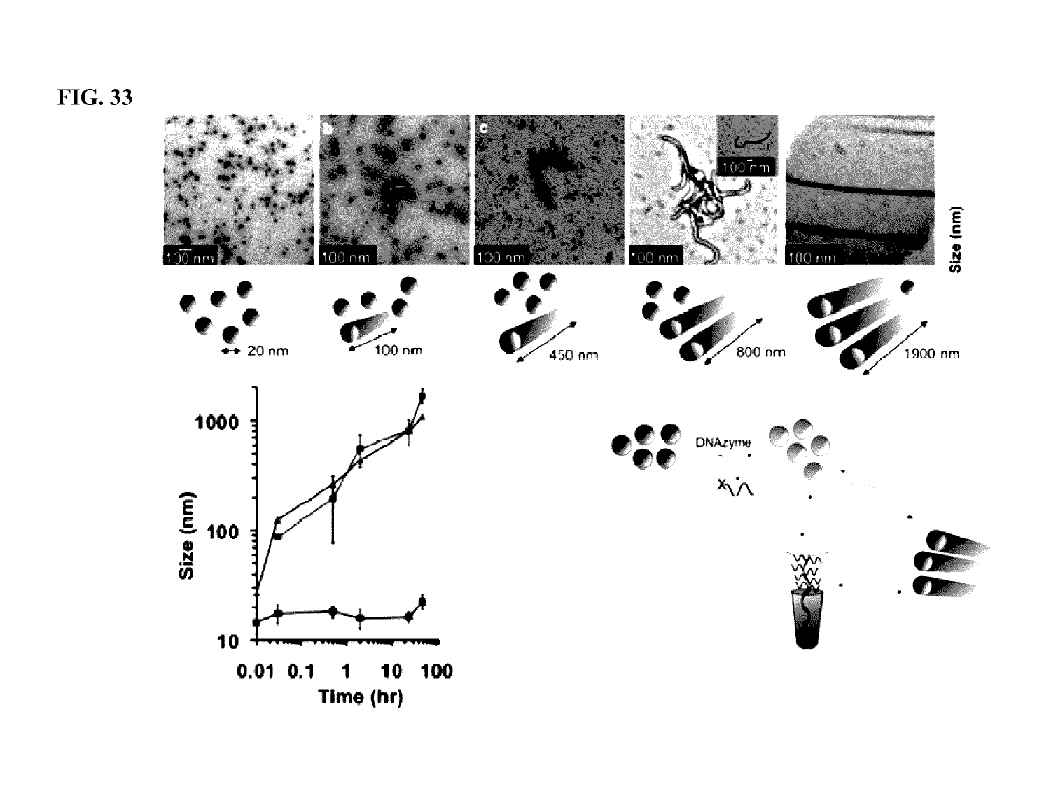

Changes to nanoparticle morphology such as cylinders (fibers) and spheres was created by DNA-based enzyme-responsive micelles. Enzyme directed cleavage of specific DNA sequences in the hydrophilic DNA brush and hydrophobic particle core of the micelle enabled a spherical nanoparticle to become a cylindrical particle (FIG. 30). Annealing of specific DNA sequences of the hydrophilic brush converted a cylindrical particle to a more spherical particle. Denaturing the DNA strands reversibly converted the spherical nanoparticle back into a cylinder (FIG. 30). FIG. 31 illustrates an increase in fluorescence of fluorescent DNA-polymer micelles (substrate) in the presence of enzyme (DNAzyme). FIG. 32 illustrates fluorescence in the DNA-polymer nanoparticles during morphological changes induces by DNAzymes and the DNA sequence in the particle shell. FIG. 33 shows that the length of the nano fibers can be controlled by the nanomaterial. At room temperature, fiber formation takes several days to go to completion. The DNA particle shell is truncated within 15 minutes when the micelles are incubated in the presence of DNAzyme.

### Example 4

### Programming Morphology of Nanoscale Polymeric Materials Nanoparticles as Diagnostics and Therapeutics

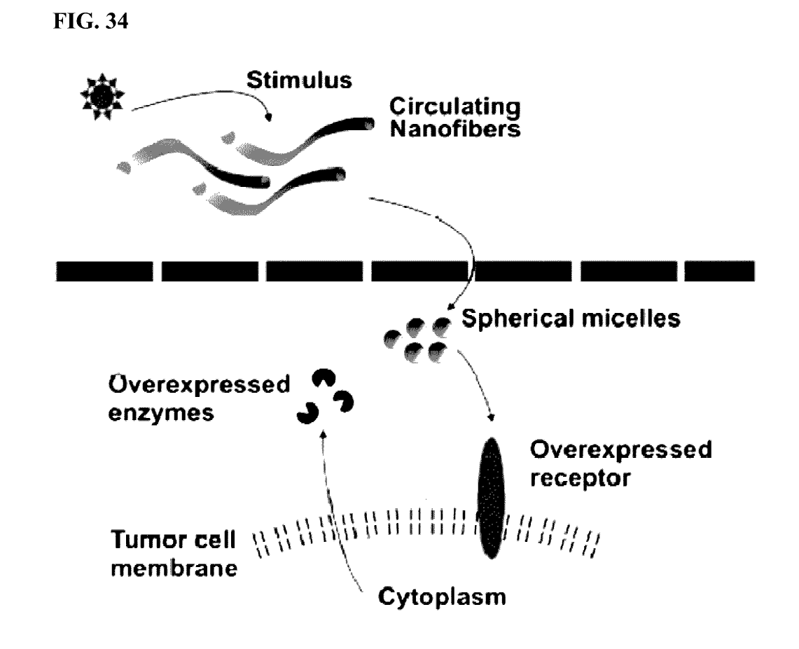

It is known that the morphology of a nanoscale materials greatly affects its properties such as circulation time, cell uptake and internalization, mode of encapsulation and mode of release of payload and imaging properties. We combined critical function not currently possible within single systems of fixed morphology and architecture. For instance, some goals of a nanomaterial are to evade macrophage uptake, circulate for many days in the body, and switch shape to enter cells. In one possible scenario (FIG. 34), circulating nanomaterials are fibers upon exposure to stimulus. In the presence of overexpressed enzymes that are due to a disease state, the nanofibers become spherical micelles. In some instances, the spherical micelles target a tumor cell that overexpresses a receptor. The micelles can be designed to carry and release drug payloads upon cell targeting or entry into a tumor cell.

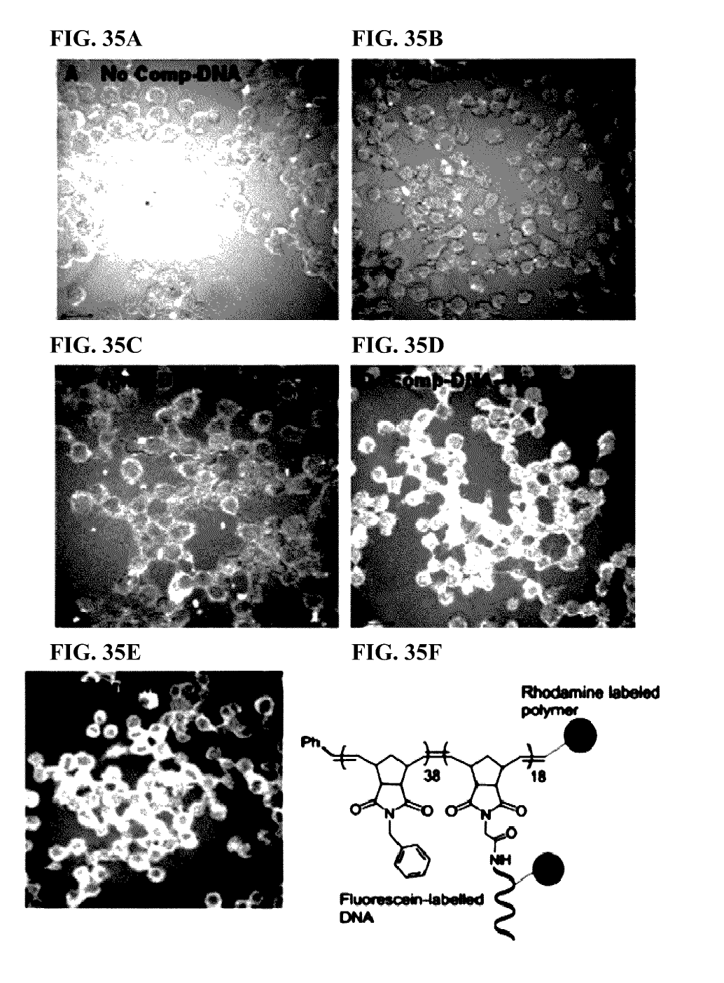

We performed an in vitro study of fibers and spheres to evaluate switchable macrophage uptake and showed that macrophage uptake is dependent on nanoparticle shape and size. Using J77 murine macrophage cells, we add either 0.1 nmole rhodamine and fluorescein co-labeled DNA-polymer nanofibers (FIGS. 35A-35D) or nanospheres (FIG. 35E) to the cells. Then, we added 0.1 nmole of complementary DNA from another 1 hr (FIG. 35B), 2 hrs (FIG. 35C) and 4 hrs (FIG. 35D). The cells were fixed, washed with PBS, and detected fluorescence by imaging. FIG. 35F also shows a schematic diagram of the DNA-polymer nanomaterial. In the absence of complementary DNA, there is no detectable fluorescent signal in the fiber sample due to FRET. Fluorescein fluorescence is robust after 4 hours of complementary DNA (FIG. 35D). In the sphere sample, fluorescence is detectable in the absence of complementary DNA (FIG. 35E). This shows the conformational differences of the nanoparticle morphologies.

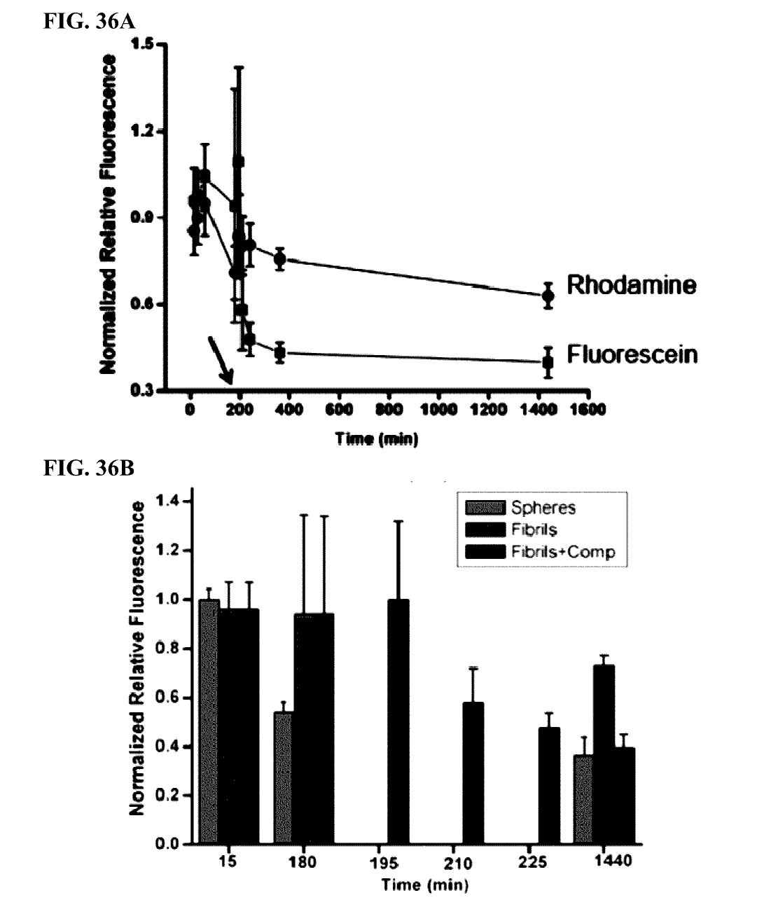

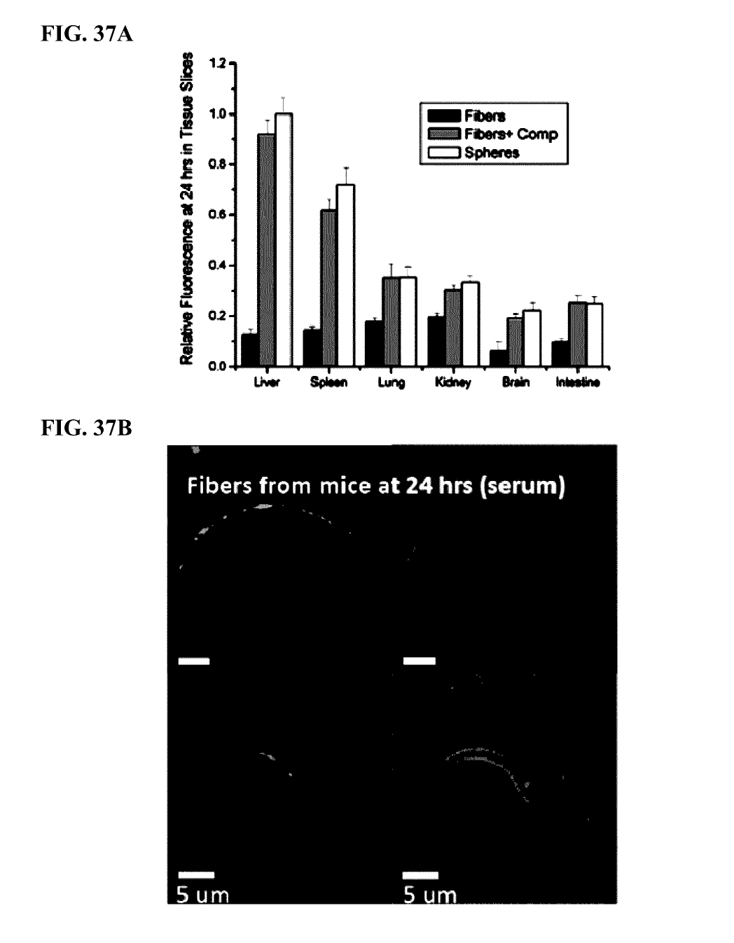

We performed an in vivo study to determine the pharmacokinetics of different nanomaterial shapes. Several groups of mice underwent tail vein injection of rhodamine and fluorescein labeled DNA-polymer spherical and fiber structures. In particular, 1 nmole of a DNA-polymer fibers mixture as injected. 1 nmole (10 μM) of complementary DNA was injected 3 hours after the initial injection. Approximately 0.5-0.1 μM of the nanoparticle mixture was present in the blood stream of each test animal. Fluorescence was monitored (FIGS. 36A-36B). After 24 hours, two mice were sacrificed and tissue dissection performed. Fluorescent nanomaterials were detected in liver, spleen, lung, kidney, brain and intestine (FIG. 37A). The relative fluorescence varied among tissue and nanomaterial morphology. Fibers were detected in the blood stream 24 hours after injection (FIG. 37B).

All publications, websites, databases, patents, and patent applications cited in this specification are incorporated by reference in their entireties for all purposes.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound comprising:
a polymer of monomers forming a first portion of monomers and a second portion of monomers, said first portion of monomers each conjugated to a hydrophobic moiety through a first linker, and said second portion of monomers each conjugated to a hydrophilic moiety through an enzyme cleavable moiety;
wherein said first portion of monomers or second portion of monomers further comprise a visualizable label, a therapeutic agent, or a targeting moiety.

2. The compound of claim 1, wherein the visualizable label is a fluorophore, a contrast agent as a reporter, or a combination thereof.

3. The compound of claim 1, wherein the therapeutic agent is selected from the group consisting of: doxorubicin, paclitaxel, cisplatin, a tyrosine kinase inhibitor, a topoisomerase inhibitor, an alkylating agent, an anthracycline, and a chemotherapeutic agent.

4. The compound of claim 1, wherein the hydrophilic moiety is a peptide-based sequence or a nucleic acid-based sequence.

5. A method of detecting diseased tissue in a subject comprising:
(a) administering a compound to the subject;
wherein the compound comprises a hydrophilic polymer with an enzyme cleavable moiety and a visualizable label;
wherein the enzyme cleavable moiety is cleaved by an enzyme present in a diseased tissue, and
wherein cleavage of the enzyme cleavable moiety in the compound causes the compound to self-assemble into an amphiphilic polymer aggregate comprising an amphiphilic polymer and the visualizable label; and
(b) determining the presence or absence of amphiphilic polymer aggregate in a tissue in the subject, wherein the presence of amphiphilic polymer aggregate in the tissue indicates that the tissue is diseased; and
(c) detecting diseased tissue in the subject where amphiphilic polymer aggregate is present.

6. (canceled)

7. The method of claim 5, wherein the enzyme is a matrix metalloprotease or matrix metalloproteinase (MMP).

8. (canceled)

9. The method of claim 5, wherein the enzyme is a protease or a nuclease.

10. The method of claim 5, wherein the enzyme cleavable moiety is a peptide structure or a DNA structure.

11. (canceled)

12. The method of claim 5, wherein the diseased tissue is a MMP-mediated malignancy, sarcoma, or metastasis.

13. The method of claim 5, wherein the visualizable label is a fluorophore, Gd3+ reporter, or a combination thereof.

14. The method of claim 5, wherein the amphiphilic polymer aggregate forms a micelle.

15. A method of treating a subject having cancer, the method comprising:
(a) administering a compound to the subject;
wherein the compound comprises a hydrophilic polymer with an enzyme cleavable moiety, a therapeutic agent, and a targeting moiety,
wherein the enzyme cleavable moiety is cleaved by an enzyme present in a diseased tissue, and;
wherein cleavage of the enzyme cleavable moiety in the compound causes the compound to self-assemble into an amphiphilic polymer aggregate acting to target the material to the tissue, and where the aggregate comprises polymeric material and the therapeutic agent;
(b) allowing a targeting moiety to localize the compound to a targeted tissue in the subject as directed by the targeting moiety in addition to enzymatically-driven aggregation targeting; and
(c) treating a subject with cancer in the subject by exposing the targeted tissue to the amphiphilic polymer aggregate containing a therapeutic agent.

16. The method of claim 15, wherein the enzyme cleavable moiety is cleaved by a protease, a nuclease, a matrix metalloprotease or matrix metalloproteinase (MMP).

17. (canceled)

18. The method of claim 15, wherein the cancer is a MMP-mediated malignancy, sarcoma, or metastasis.

19. The method of claim 15, wherein the enzyme is a protease, nuclease, matrix metalloprotease, or MMP.

20-21. (canceled)

22. The method of claim 15, wherein the therapeutic agent is selected from the group comprising doxorubicin, paclitaxel, cisplatin, a tyrosine kinase inhibitor, a topoisomerase inhibitor, an alkylating agent, an anthracycline, and a chemotherapeutic agent.

23. The method of claim 15, wherein the amphiphilic polymer aggregate forms a micelle.

24. The method of claim 15, wherein the targeting moiety is peptide-based sequence or a nucleic acid-based sequence.

25. An enzymatically-generated, self-assembled amphiphilic polymer aggregate comprising: an amphiphilic polymer comprising a hydrophilic cryptic-amphiphile associated with a polypeptide cleaved by a matrix metalloproteinase, and a visualizable label.

26. An enzymatically-generated self-assembled amphiphilic polymer aggregate comprising: an amphiphilic polymer comprising a hydrophilic cryptic-amphiphile associated with a nucleic acid structure cleaved by an enzyme, and a visualizable label.

27-28. (canceled)

29. The compound of claim 1, said polymer further comprising a third portion of monomers each conjugated to a non-enzyme cleavable hydrophilic moiety.

30. The compound of claim 29, wherein said non-enzyme cleavable hydrophilic moiety is polyethylene glycol (PEG) or a visualizable label.

31. The compound of claim 1, wherein said enzyme cleavable moiety is protease cleavable.

32. The compound of claim 1, wherein said enzyme cleavable moiety is nuclease cleavable.

33. The compound of claim 1, wherein enzymatic cleavage of said enzyme cleavable moiety causes said compound to form a micelle.
